(19) United States
(12) Patent Application Publication
(10) Pub. No.: US 2004/0067198 A1
(43) Pub. Date: Apr. 8, 2004

(54) Emissive multichromophoric systems

(76) Inventor: Michael J Therien, Philadelphia, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/467,107

(22) PCT Filed: Feb. 26, 2002
(86) PCT No.: PCT/US02/05584

(51) Int. Cl. A61K 49/00

(52) U.S. Cl. 424/9.61

(57) ABSTRACT

Synthetic multichromophoric systems exhibiting low energy fluorescent excited states in which the transition dipoles of the pigment building blocks are correlated in defined phase relationships are provided. The polarized nature of these singlet excited states can be maintained over long (ns) timescales. In preferred embodiements ethyne- and butadiyne-bridged multiporphyrin species that manifest high excited-state anisotropies display exceptionally large emitting dipole strengths, establishing a new precedent for superradiant oligopigment assemblies.

### FIELD OF THE INVENTION

This invention relates to synthetic multichromophoric systems that preferably exhibit: (i) low energy emissive excited states in which the transition dipoles of the constituent pigment building blocks are correlated in defined phase relationships, (ii) excited state polarization over long timescales, (iii) emission quantum yields that have an unusual dependence upon supramolecular structure and emission wavelength, (iv) collective oscillator behavior in their respective electrochemically excited states, and (v) integrated emission oscillator strengths that are large with respect to that manifest by the benchmark monomeric chromophore.

### BACKGROUND OF THE INVENTION

The desire to enhance superradiant emission and electroluminescence in processable materials has generated considerable interest in the photophysical properties of broad classes of conjugated oligomers and polymers. (See, e.g., U.S. Pat. No. 5,798,306, which is incorporated by reference). Interestingly, these technologically important electrooptic properties appear to be connected, in that they both derive from long-range electronic excitations (one-dimensional excitons) that extend over multiple monomer units. Although electroluminescence in conjugated polymers has been a subject of long-standing interest, detailed examination of the superradiant properties of these materials has come to the fore only recently, fueled by the observation that amplification of stimulated emission (ASE) results when thin films of superradiant polymers are optically pumped at high intensity.

Superradiance is an example of cooperative emission that originates when an ensemble of emitters (emissive pigment blocks) is excited into a correlated state that possesses a macroscopic dipole moment. One key hallmark of this optical, nonlinear phenomenon is the emission of a coherent radiation pulse with a peak intensity proportional to the square of the number of correlated emitters. Supperradiant pigment arrays thus manifest radiative rate constants kᵣ that exceed that determined for their respective monomeric chromophoric building blocks, a dependence highlighted in the Einstein equation for spontaneous emission (eq 1),

$${k}_{r} = \frac{16 {π}^{3}}{3 {ϵ}_{o} h {c}^{3}} [ \frac{{n}^{3}}{{ϵ}_{r}} ] {E}^{3} {\vert ⟨ μ ⟩ \vert}^{2} \qquad ( 1 )$$

where E is the emission energy, n and ε are respectively the medium's refractive index and dielectric strength, and <μ> is the emission transition dipole moment. Note that the magnitude of kᵣ is a signature of the number of coupled oscillators; hence, for an assembly of m pigments exhibiting superradiant emission, the classically predicted, maximal value of kᵣ that could be observed corresponds to m times the radiative rate constant determined for the corresponding monomeric chromophore.

In one aspect of the present invention, the monomeric chromophoric building blocks are conjugated to form dimers, trimers, oligomers or polymers. The monomeric chromophoric building blocks can, for example, be porphyrins. Those in the art will recognize that porphyrins are derivatives of porphine, a conjugated cyclic structure of four pyrrole rings linked through their 2- and 5-positions by methine bridges. Porphyrins can bear up to 12 substituents at meso (i.e. α) and pyrrolic (i.e.,β) positions thereof. (See, e.g., U.S. Pat. Nos. 5,371,199, 5,783,306, and 5,986,090 which are incorporated by reference) Porphyrins can be covalently attached to other molecules. The electronic features of the porphyrin ring system can be altered by the attachment of one or more substituents. The term “porphyrin” includes derivatives wherein a metal atom is inserted into the ring system, as well as molecular systems in which ligands are attached to the metal. The substituents, as well as the overall porphyrin structure, can be neutral, positively charged, or negatively charged.

Numerous porphyrins have been isolated from natural sources. Notable porphyrin-containing natural products include hemoglobin, the chlorophylls, and vitamin B12. Also, many porphyrins have been synthesized in the laboratory, typically through condensation of suitably substituted pyrroles and aldehydes. However, reactions of this type generally proceed in low yield, and cannot be used to produce many types of substituted porphyrins.

### SUMMARY OF THE INVENTION

In one aspect, the present invention conjugated multichromophoric systems including a polymer comprising a plurality of linked porphyrinic monomer units having formula (1), (2), or (3):

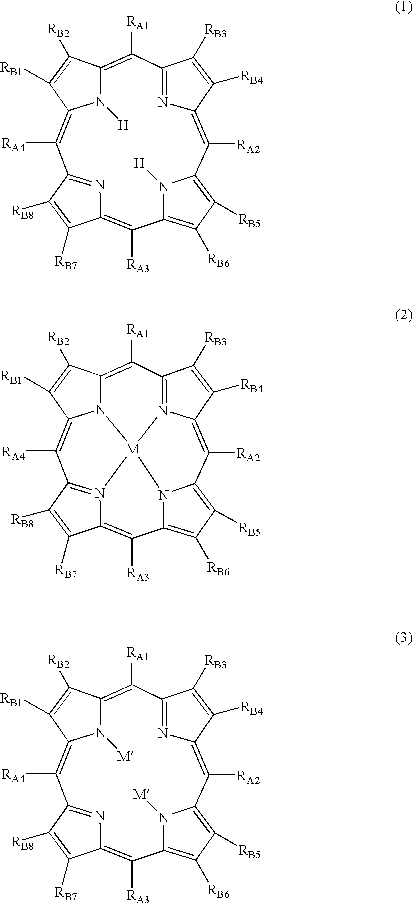

wherein M and M′ are metal atoms and R_{A1}—R_{A4} and R_{B1}—R_{B8} are, independently, H or chemical functional groups that can bear a negative charge prior to attachment to a porphyrin compound. In certain embodiments, at least one of R_{A1}—R_{A4} has formula CH═CH2 or at least one of R_{A1}—R_{A4} or R_{B1}—R_{B8} has formula C(R_{C})═C(R_{D})(R_{E}), provided that at least one of R_{C}, R_{D}, and R_{E} is not H, where R_{C}, R_{D}, and R_{E} are, independently, H, F, Cl, Br, I, alkyl or heteroalkyl having from 1 to about 20 carbon atoms, aryl or heteroaryl having about 4 to about 20 carbon atoms, alkenyl or heteroalkenyl having from 1 to about 20 carbon atoms, alkynyl or heteroalkynyl having from 1 to about 20 carbon atoms, trialkylsilyl or porphyrinato; M is a transition metal, a lanthanide, actinide, rare earth or alkaline metal. R_{C}, R_{D}, and R_{E} also can include peptides, nucleosides, and/or saccharides.

In other embodiments, at least one of R_{A1}—R_{A4} or R_{B1}—R_{B8} has formula C≡C(R_{D}). In further preferred embodiments, at least one of R_{A1}—R_{A4} is haloalkyl having from 1 to about 20 carbon atoms. In further preferred embodiments, at least one of R_{B1}—R_{B8} is haloalkyl having 2 to about 20 carbon atoms or at least at least five of R_{B1}—R_{B8} are haloalkyl having from 1 to about 20 carbon atoms or haloaryl having from about 6 to about 20 carbon atoms. In further preferred embodiments, at least one of R_{B1}—R_{B8} is haloaryl or haloheteroaryl having about 4 to about 20 carbon atoms. In still further preferred embodiments, at least one of R_{A1}—R_{A4} or R_{B1}—R_{B8} includes an amino acid, peptide, nucleoside, or saccharide.

The present invention also provides processes and intermediates for preparing substituted porphyrins. In certain embodiments, the processes comprise providing a porphyrin compound having formula (1), (2), or (3) wherein at least one of R_{A1}—R_{A4} or R_{B1}—R_{B8} is a halogen and contacting the porphyrin compound with a complex having formula Y(L)₂ wherein Y is a metal and L is a ligand. This produces a first reaction product, which is contacted with an organometallic compound having general formula T(R_{L})_{z}(R_{O}), T(R_{L})_{z}(R_{O})_{y}(X_{B})w, T(R_{O})(X_{B}) or T(R_{O}) y where T is a metal; X_{B} is a halogen; R_{L} is cyclopentadienyl or aryl having about 6 to about 20 carbon atoms; R_{O} is alkyl having 1 to about 10 carbon atoms, alkenyl or alkynyl having 2 to about 10 carbon atoms, aryl having about 6 to about 20 carbon atoms; z and w are greater than or equal to 0; and y is at least 1. This contacting produces a second reaction product which, through reductive elimination, yields a third reaction product that contains a porphyrin substituted with R_{O}.

In another aspect, the invention provides polymers comprising linked porphyrin units. In certain embodiments, porphyrin units having formula (1), (2), or (3) share covalent bonds. In other embodiments at least one of R_{A1}—R_{A4} or R_{B1}—R_{B8} function as linking groups. In these embodiments, at least a portion of a linking group can have formula [C(R_{C})═C(R_{D})(R_{E})]ₓ, [C≡C(RD)]ₓ, [CH2(R_{C})—CH(R_{D})(R_{E})]ₓ or [CH═CH(R_{D})]ₓ where x is at least 1. The remaining of R_{A1}—R_{A4} and R_{B1}—R_{B8} can be H, halogen, alkyl or heteroalkyl having 1 to about 20 carbon atoms or aryl or heteroaryl having 4 to about 20 carbon atoms, C(R_{C})═C(R_{D})(R_{E}), C≡C(R_{D}), or a chemical functional group that includes a peptide, nucleoside, and/or saccharide. In other preferred embodiments, the linking group is cycloalkyl or aryl having about 6 to about 22 carbon atoms.

The invention also provides processes for preparing porphyrin-containing polymers. In certain embodiments, the processes comprise providing at least two compounds that, independently, have formula (1), (2) or (3) wherein at least one of R_{A1}—R_{A4} or R_{B1}—R_{B8} in each of the compounds contains an olefinic carbon-carbon double bond or a chemical functional group reactive therewith. In other embodiments, at least one of R_{A1}—R_{A4} or R_{B}—R_{B8} in each of the compounds contains a carbon-carbon triple bond or a chemical functional group reactive therewith. The compounds are then contacted for a time and under reaction conditions effective to form covalent bonds through the carbon-carbon double and/or triple bonds.

In another aspect of the invention, emissive pigment building blocks such as porphyrin monomer units that, independently, have formula (1), (2), or (3), are linked to form a conjugated dimer, trimer, oligomer, polymer, or other highly conjugated synthetic multichromophoric systems that exhibits low energy fluorescent excited states in which the transition dipoles of the pigment building blocks are correlated in defined phase relationships. Analyses of corresponding fluorescence intrinsic decay rate and quantum yield data show that, in another embodiment, ethyne- and butadiyne-bridged multiporphyrin species that manifest high excited-state anisotropies display exceptionally large emitting dipole strengths, establishing a new precedent for superradiant oligopigment assemblies. This photophysical behavior derives not only from the fact that these conjugated pigment arrays behave as collective oscillators; the large transition dipole moment of the porphyrinic monomer unit combined with strong chromophore-chromophore electronic coupling ensure large Frank-Condon barriers for intersystem crossing between their respective S₁ and T₁ states. These results indicate that substantial emitting dipole strengths can in fact be realized for low energy fluorescing chromophores, and that simple energy gap law considerations do not preclude the design of high quantum yield near IR emitters.

In another aspect, the present invention provides methods comprising the steps of providing a conjugated compound comprising at least two covalently bound moieties and then exposing the compound to an energy source for a time and under conditions effective to cause it to emit light that has a wavelength of 650-2000 nm and is of an intensity that is greater than a sum of light individually emitted by the component moieties. In preferred embodiments emission from said materials can be effected by optical or electrical pumping. For example, when these materials are optically pumped, evaluation of the emission dipole strength can be made from determination of the emission quantum yield and the corresponding emission decay rate using conventional methods [see for example: Lakowicz, J. R. Principles of Fluorescence Spectroscopy (Plenum Press, New York, 1983); Turro, N. J. Modern Molecular Photochemistry (The Benjamin/Cummings Publishing Co., Inc., Menlo Park, 1978); Dexter, D. L. J. Chem. Phys. 21, 836-850 (1953); Dicke, R. H. Phys. Rev. 93, 99 (1954)].

For example, the fluorescence quantum yield (QY) can be determined by the reference method[Lakowicz, 1983], using the above relation where ∫I_{complex} and ∫I_{standard} are the respective, total integrated fluorescence intensities of the complex and emission standard, A_{complex} and A_{standard} are the corresponding wavelength-specific absorbances, and QY_{standard} is the accepted fluorescence QY value for the standard chromophore. The quantity (n_{0complex}/n_{0standard})² represents the solvent refractive index correction. Steady state emission spectra can be obtained on a conventional luminescence spectrometer having the appropriate emission detectors. Sample concentrations are adjusted typically such that the absorbance is between 0.005 and 0.04 at the excitation wavelength. Emission spectra obtained for the chromophore (fluorophore, phosphore, or lumophore) are corrected to account for the wavelength-dependent efficiency of the detection system which can be determined using the spectral output of a calibrated light source obtained from the National Bureau of Standards. Secondary corrections to the emission spectra used to determine QYs (such as energy-dependent intensity corrections necessitated by the variable band pass/constant wavelength resolution data acquisition mode of the grating monochromator) are performed as outlined by Lakowicz[Lakowicz, 1983]. Quantum yields are determined typically using two standard benchmarks for each chromophore.

Emitting dipole strengths are defined as <μ>_{chromophore}/<μ>_{reference}, where <μ>_{reference} corresponds to the emission dipole strength of one of the covalently bound moieties that defines the conjugated compound. <μ> values can be determined from the Einstein equation for spontaneous emission, when the radiative rate constant kᵣ has been determined from appropriate time-resolved spectroscopic techniques [Lakowicz, J. R. Principles of Fluorescence Spectroscopy (Plenum Press, New York, 1983); Turro, N. J. Modern Molecular Photochemistry (The Benjamnin/Cummings Publishing Co., Inc., Menlo Park, 1978); Dexter, D. L. J. Chem. Phys. 21, 836-850 (1953); Dicke, R. H. Phys. Rev. 93, 99 (1954)].

In certain embodiments, the compound exhibits an integral emission oscillator strength that is greater than a sum of emission oscillator strengths exhibited by its component moieties. Representative moieties are those that include a conjugated ring system. Preferably, at least one of the moieties is a laser dye, fluorophore, lumophore, or phosphore. Particularly preferred moieties include porphyrins, porphycenes, rubyrins, rosarins, hexaphyrins, sapphyrins, chlorophyls, chlorins, phthalocyanines, porphyrazines, bacteriochlorophyls, pheophytins, texaphyrins, and their corresponding metalated derivatives. Another class of representative moieties are conjugated macrocycles comprising 16 or more carbon atoms and four or more heteroatoms such as N, O, S, Se, Te, B, P, As, Sb, Si, Ge, Sn, and Bi.

The moieties preferably are bound by at least one carbon-carbon double bond, carbon-carbon triple bond, a combination thereof, or an imine, phenylene, thiophene, amide, ether, thioether, ester, ketone, sulfone, or carbodiimide group. Representative bond types include ethynyl, ethenyl, allenyl, butadiynyl, polyvinyl, polyynyl, thiophenyl, furanyl, pyrrolyl, p-diethynylarenyl bonds and any conjugated hetrocycle that bears diethynyl, di(polyynynyl), divinyl, di(polyvinvyl), or di(thiophenyl) substituents. Such materials thus include, laser dyes, fluorophores, lumophores, and/or phosphore that are covalently bound with, for example, alkynyl or alkenyl bonds.

The conjugated synthetic multichromophoric systems of the invention can be used, for example, as dyes, catalysts, contrast agents, antitumor agents, antiviral agents, electroluminescent materials, LEDs, lasers, photorefractive materials and in chemical sensors and electrooptical devices. Thus, in one aspect, the present invention provides lasers in which a dye solution is disposed in a resonant cavity and comprises a compound of the invention and an aqueous or non-aqueous solvent that is substantially unable to chemically react with said compound and to absorb and emit light at a wavelength at which said compound absorbs and emits light. Lasers according to the invention further include a pumping energy source that produces stimulated emission in the dye solution.

Further lasers according to the invention are those that include a solid body that, in turn, includes a compound of the invention and a host polymer that is unable to chemically react with the compound and unable to absorb and emit light at a wavelength at which the compound absorbs and emits light. Such lasers further include an energy source that either is coupled with the solid body and generates light in the solid body, or is coupled with the host polymer and generates light therein. Also, an optical amplifier comprising a polymeric optical waveguide and a compound of the invention is provided.

The present invention also provides polymer grids comprising a body of electrically conducting organic polymer. Such a body has an open and porous network morphology and defines an expanded surface, area void-defining porous network. An active electronic material comprising a compound of the invention is located within at least a portion of the void spaces defined by the porous network. The conducting organic polymer may also include a compound of the invention.

The present invention also provides polymer grid electrodes comprising a body of electrically conducting organic polymer that is electrically joined to an electrical connector. The body should have an open and porous network morphology and define an expanded surface area, void-defining porous network, with an active electronic material comprising a compound of the invention located within at least a portion of the void spaces defined by the porous network.

The invention also provides solid state polymer grid triodes comprising a first electrode and a second electrode spaced apart from one another with a polymer grid comprising a body of electrically conducting organic polymer that includes a compound of the invention. The body preferably has an open and porous network morphology and defines an expanded surface area void-defining porous network interposed between the first electrode and the second electrode.

In another aspect, the present invention provides light-emitting polymer grid triodes comprising a first electrode and a second electrode spaced apart from one another with a polymer grid comprising a body of electrically conducting organic polymer. The body in such a triode has an open and porous network morphology and defines an expanded surface area, void-defining porous network interposed between the first and second electrodes. An active luminescent semiconducting electronic material comprising a compound of the invention is interposed between the first and second electrodes, and serves to transport electronic charge carriers between the first and second electrodes, the carriers being affected by the polymer grid such that on applying a turn-on voltage between the first and second electrodes, charge carriers are injected and light is emitted.

The present invention also relates to light-responsive diode systems comprising a diode that, in turn, includes: a conducting first layer having high work function; a semiconducting second layer in contact with the first layer, the second layer made comprising a compound of the invention; and a conducting third layer in contact with the second layer. Systems according to the invention further include a source for applying a reverse bias across the diode, a source for impinging light upon the diode, and a source for detecting an electrical current produced by the diode when the reverse bias is applied to the diode and light is impinged upon the diode.

The present invention also provides light-responsive diode systems that comprise a diode that itself includes: a conducting first layer having high work function; a semiconducting second layer in contact with the first layer, the second layer made comprising a compound of the invention; and a conducting third layer in contact with the second layer, the third layer comprising an inorganic semiconductor doped to give rise to a conductive state. Such systems further include a source for applying a reverse bias across the diode, a source for impinging light upon the diode, and a source for detecting an electrical current produced by the diode when the reverse bias is applied to the diode and light is impinged upon the diode.

Also provided are dual function light-emitting, light responsive input-output diode systems comprising a diode having a conducting first layer having high work function, a semiconducting second layer in contact with the first layer comprising a compound of the invention, and a conducting third layer in contact with the second layer. Such systems further comprise a source for applying a reverse bias across the diode, a source for impinging light upon the diode, and a source for detecting an electrical current produced by the diode when the reverse bias is applied to the diode and light is impinged upon the diode.

The present invention also provides dual function light-emitting, light responsive input-output diode systems comprising a diode having a conducting first layer having high work function, a semiconducting second layer in contact with the first layer that comprises a compound of the invention, and a conducting third layer in contact with the second layer. Such systems further include a source for applying a reverse bias across the diode, a source for impinging an input signal or light upon the diode, a source for detecting an electrical current produced by the diode when the reverse bias is applied to the input signal of light is impinged upon the diode, a source for halting the applying of reverse bias, and a source for applying a positive bias output signal across the diode, the positive bias output signal being adequate to cause the diode to emit an output signal of light.

The invention a provides dual function input-output processes comprising the steps of applying a reverse bias across the diode and impinging an input signal of light upon the diode, detecting as an electrical input signal an electrical current or voltage produced by the diode when the reverse bias is applied to the diode and the input signal of light is impinged upon the diode, halting the applying of reverse bias, and applying a positive bias output signal across the diode, the positive bias output signal being adequate to cause the diode to emit an output signal of light in response thereto.

Also provided are articles comprising a unitary solid state source of electromagnetic radiation, in which the source has a layer structure that comprises a multiplicity of layers, including two spaced apart conductor layers with compound of the invention therebetween, and further comprising contacts for causing an electrical current to flow between the conductor layers, such that incoherent, electromagnetic radiation of a first wavelength is emitted from the compound of the invention. The layer structure preferably comprises an optical waveguide comprising a first and a second cladding region with a core region therebetween, with the optical waveguide disposed such that at least some of said incoherent electromagnetic radiation of the first wavelength is received by the optical waveguide, and the core region comprises a layer of a second organic material selected to absorb the incoherent electromagnetic radiation of the first wavelength, and to emit coherent electromagnetic radiation of a second wavelength, longer than the first wavelength, in response to the absorbed incoherent electromagnetic radiation.

The present invention also provides methods for screening compounds. In preferred embodiments, such methods comprise the steps of providing a conjugated compound comprising at least two covalently bound moieties; exposing the compound to an energy source for a time and under conditions effective to cause it to emit light that has a wavelength of 650-2000 nm; and determining whether or not that emitted light is either (1) of an intensity that is greater than a sum of light emitted individually by the moieties or (2) larger than emitted by either of the covalently bound moieties.

Also provided are methods in which a compound of the invention is attached to a targeting agent which provides localization of the compound in select body tissues. A probe light source can be held external to the tissue to excite the compound into an emissive state that has significant emission dipole strength in the 700-1100 nm spectral domain.

### BRIEF DESCRIPTION OF THE FIGURES

The numerous objects and advantage of the present invention can be better understood by those skilled in the art by reference to the accompanying figures, in which:

FIG. 1 shows conjugated porphyrin arrays (compounds 4-12), as well as electronic absorption spectra thereof. Uncorrected emission spectra are shown as insets. Solvent (Compounds 4-10)=CHCI₃; solvent (compounds 11-12)=10:1 CHCl₃:pyridine.

FIG. 2 shows anisotropic fluorescence dynamics of compounds 4-6. (A) Comparative time dependent decays for the fluorescence polarized parallel and perpendicular to the polarization of the exciting light. (B) Time dependence of the fluorescence anisotropy. Excitation (λₑₓ) and emission (λₑₘ) wavelengths, along with fluorescence lifetime and anisotropy depolarization time constants are listed Table 1.

FIG. 3 is a schematic of (A) Potential energy diagram illustrating the dependence of the magnitude of electronic state energy separation and the extent of equilibrium nuclear displacement (ΔQ) upon vibrational wave function overlap. (B) Potential energy diagrams highlighting the effect of increasing S₁—T₁ nuclear displacement upon the magnitude of intersystem crossing rate constants k_{ISC}.

FIG. 4 shows photophysical properties of S₁-excited states of (porphinato)zinc(II) Complexes 1-12, including (5-ethynyl-10,20-diphenylporphinato)zinc(II) (Compound 1), (5,15-diethynyl-10,20-diphenylporphinato)zinc(II) (Compound 2), and (2-ethynyl-5,10,15,20-tetraphenylporphinato)zinc(II) (Compound 3): fluorescence lifetime and time-resolved anisotropy data.^{a-d}

(a) Samples for transient spectroscopic studies were kept rigorously dry using standard inert-atmosphere techniques; all data presented were recorded at 293 K in 10:1 CHCl₃:pyridine.

(b) The fluorescence lifetimes were determined using a time-correlated single-photon counting (TCSPC) apparatus (Regional Laser and Biotechnology Laboratory, University of Pennsylvania) that has been described previously; instrument response function=20 Ps fwhm. Data were analyzed using the Lifetime (RLBL) program. Compounds 1-12 exhibit monoexponential decays of the isotropic fluorescence; the average evaluated _{χ}² value from fitting of these data for 1-12 was 1.06±0.08.

(c) Time-resolved anisotropy decay data were obtained using rotating polarization filters to alternatively select the parallel and the perpendicular components of the emission; all other experimental procedures were identical to the lifetime measurements. Rotational correlation times were calculated using the method outlined by Wahl, Holtom (Holtom, G. R. Proc. SPIE-Int. Soc. Opt. Eng. 1204, 2-12 (1990); Wahl, P. Biophys. Chem. 10, 91-104 (1979)). λ_{cx} and λₑₘ denote respectively the excitation and emission probe wavelengths. All anisotropy decays could be fit as simple monoexponential decay processes, (\overline{_{χ}²} (1-12)=1.07±0.05).

(d) τ_{F}=fluorescence lifetime; r_{O}=initial fluorescence anisotropy determined 20 ps after excitation; τᵣ=rotational diffusional time constant.

(e) r_{O} values determined 20 ps after excitation.

(f) Determined by van Grondelle (Monshouwer, R., Abrahamsson, M., van Mourik, F. & van Grondelle, R. J. Phys. Chem. B 101, 7241-7248 (1997)).

(g) r_{O} determined 8 ps following excitation for Bchl a, and at zero time for B820, LH-2, and LH-1.

FIG. 5 shows Fluorescence Quantum Yield, Stokes Shift, and Calculated Transition Dipole Moment Data of Conjugated [(Porphinato)zinc] Complexes 1-12.

(a) The fluorescence quantum yield (QY) of these species was determined by the reference method, using the relation:

$${QY}_{complex} = \frac{\int {I}_{complex} {{A}_{standard} ( {n}_{0 complex} )}^{2}}{\int {I}_{standard} {{A}_{complex} ( {n}_{0 standard} )}^{2}} {QY}_{standard}$$

where ∫I_{complex} and ∫I_{standard} are the respective, total integrated fluorescence intensities of the complex and emission standard, A_{complex} and A_{standard} are the corresponding wavelength-specific absorbances, and QY_{standard} is the accepted fluorescence QY value for the standard chromophore. The quantity (n_{0complex}/n_{0standard})² represents the solvent refractive index correction. Steady state fluorescence emission spectra were obtained on a Perkin-Elmer LS 50 Luminescence Spectrometer. The concentrations of all samples were adjusted such that their absorbance was between 0.01 and 0.04 at the excitation wavelength. All spectra were collected with the single excitation and emission monochromators set at 5 nm. Fluorescence spectra obtained for the (porphinato)zinc(II) complexes as well as the chromophores used as emission standards were corrected to account for the wavelength-dependent efficiency of the detection system which was determined using the spectral output of a calibrated light source obtained from the National Bureau of Standards. Secondary corrections to the emission spectra used to determine QYs (such as energy-dependent intensity corrections necessitated by the variable band pass/constant wavelength resolution data acquisition mode of the grating monochromator) were performed as outlined by Lakowicz. Quantum yields were determined using two standard benchmarks for each of the conjugated bis- and tris[(porphinato)zinc(II)] complexes. (Tetraphenylporphinato)zinc(II) (TPPZn) (QY=0.033, benzene) served as the reference porphyrinic fluorescence emitter, while a standard laser dye that featured significant emission profile overlap with that of the unknown complex served as a secondary reference. Benchmark fluorescence emitters utilized were [dye (QY; solvent; λₑₘ(nm))]: (i) TPPZn (0.033; benzene; (598, 647)); (ii) TPPZn (0.028; 10:1 CHCl₃:pyridine; (613, 661)); (iii) DODCI (0.44; EtOH; (605)); (iv) HITCI (0.28; MeOH (59); (660)); (v) IR-125 (0.13; DMSO; (826)); (vi) DTDCI (0.78; DMSO; (684)). When TPPZn was used as the standard fluorescence emitter, excitation of both the unknown and reference was carried out at either 400 or 428 nm. When DODCI, HITCI, IR-125, and DTDCI dyes were utilized as emission reference compounds, λₑₓ corresponded to a wavelength within the Q-state manifold of compounds 1-12. As a check for internal self consistency, the QY for each emission standard was experimentally evaluated by the reference method in which another laser dye served as the benchmark fluorescence emitter. In all such experiments, the computed QY was always within ±10% of established literature value, confirming the appropriateness of the emission correction factors implemented throughout the 600-850 nm spectral regime in these experiments. The standard error in quantum yields determined by this method is typically taken as ±20% of the reported value. The QY entries correspond to the average of values obtained from at least three independent measurements.

(b) Transition dipole moments were calculated by integrating plots of extinction coefficient per wavenumber (ε(\overline{υ})/\overline{υ}) vs. wavenumber (\overline{υ}) using the relation μ²=(9.188×10⁻³/n₀) ∫{ε(M⁻¹cm⁻¹)(\overline{υ})/\overline{υ}}εl\overline{υ} where n₀ is the refractive index of the solvent and μ is the transition dipole moment in Debye. For these calculations, the B band region transition dipole moment corresponded to an integration carried out over the 360 to 520 nm spectral domain, while the reported Q-band value derives from an analogous integration over the 520 to 900 nm wavelength range.

(c) See R. Monshouwer, M. Abrahamsson, F. van Mourik, R. van Grondelle, J. Phys. Chem. B 101, 7241-7248 (1997).

FIG. 6 shows Comparative Radiative Lifetimes and Emitting Dipole Strengths of Conjugated Chromophores 1-12 vs. Benchmark Biological Antennae Systems.

(a) Radiative lifetimes were calculated using the relation τ_{rad}=τ_{F}/QY; QY (fluorescence quantum yield) values utilized were the average of those reported in Table II.

(b) Emitting dipole strengths=<μ>_{chromophore}/<μ>_{reference}.<μ> values were determined using eq. 5; emission energies used in this calculation correspond to the frequency that partitions the integrated emission oscillator strength into blue and red components having equivalent area. For compounds 1-12, emitting dipole strengths are referenced both to TPPZn and an appropriate benchmark ethyne-derivatized (porphinato)zinc(II) monomer.

(c) For meso-to-meso and meso-to-β bridged arrays, compound 1 served as the reference ethyne-elaborated porphyrin chromophore; β-to-β bridged compounds utilized (porphinato)zinc(II) species 3 as the conjugated pigment reference.

(d) Determined by van Grondelle.

### DETAILED DESCRIPTION OF THE INVENTION

Those skilled in the art will recognize the wide variety of dimers, trimers, oligomers or polymers that can be prepared from the porphyrin-containing compounds of the invention. For instance, somewhat linear polymer chains can be formed wherein a portion of the polymer has general formula (P_{N})ᵣ where P_{N} is a porphyrin unit and r is at least 2. In further embodiments, linear polymer chains have general formula:

[—(Q_{L})_{L}—(P_{N})ₛ—]ₕ

where Q_{L} is a linking group, P_{N} is a porphyrin unit, and h, l, and s are independently selected to be at least 1. For example, a portion of such polymers can have formula:

[—(P_{N1})_{s′}—(Q_{L1})_{l′}—(P_{N2})_{s″}—(Q_{L2})_{l—]l}

wherein P_{N1} and P_{N2} are independently selected porphyrin units, Q_{L1} and Q_{L2} are independently selected linking groups, and l′, l″, s′, and s″ are at least 1. These essentially linear polymer chains can be cross-linked such that a portion of the polymer has general formula:

[—(Q_{H})ₕ—(P_{N})ᵤ—]_{υ}

wherein Q_{H} is a linking group, and h, u, and v are independently selected to be at least 1. A portion of these cross-linked polymers can have formula:

[—(P_{N3})_{u′}—(Q_{H1})_{h′}—(P_{N1})_{u″}—(Q_{H2})_{h′}—]ᵥ

wherein P_{N3} is a porphyrin unit, Q_{H1} and Q_{H2} are independently selected linking groups, and h′, h″, u′, and u″ are at least 1. Thus, one possible cross-linked polymer has formula:

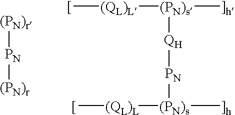

where r′ is at least 1.

The dimers, trimers, oligomers and polymers of the invention are generally formed by contacting a substituted porphyrin with a second compound containing functionality that is reactive with the functionality contained within the porphyrin. Preferably, the porphyrin contains an olefinic carbon-carbon double bond, a carbon-carbon triple bond or some other reactive functionality. The contacting should be performed under conditions effective to form a covalent bond between the respective reactive functionalities. Preferably, porphyrin-containing polymers are formed by metal-mediated cross-coupling of, for example, dibrominated porphyrin units. Also, porphyrin-containing polymers can be synthesized using known terminal alkyne coupling chemistry. (see, e.g., Patai, et al., The Chemistry of Functional Groups, Supplement C, Part 1, pp. 529-534, Wiley, 1983; Cadiot, et al., Acetylenes, pp. 597-647, Marcel Dekker, 1964; and Eglinton, et al., Adv. Org. Chem., 1963, 4, 225) As will be recognized, the second compound noted above can be a substituted porphyrin of the invention or some other moiety such as an acrylate monomer. Thus, a wide variety of copolymeric structures can be synthesized with the porphyrins of the invention. Through careful substituent selection the porphyrins of the invention can be incorporated into virtually any polymeric matrix known in the art, including but not limited to polyacetylenes, polyimides, polyacrylates, polyolefins, pohyethers, polyurethanes, polyquinolines, polycarbonates, polyanilines, polypyrroles, and polythiophenes. For example, fluorescent porphyrins can be incorporated into such polymers as end-capping groups.

The conjugated synthetic multichromophoric systems of the invention can be used, for example, as dyes, catalysts, contrast agents, antitumor agents, antiviral agents, liquid crystals, electroluminescent materials, LEDs, lasers, photorefractive materials, in chemical sensors and in electrooptical and solar energy conversion devices. They also can be incorporated into supramolecular structures. The polymers and supramolecular structures, which anchor porphyrin units in a relatively stable geometry, should improve many of the known uses for porphyrins and even provide a number of new uses, such as in a solid phase system for sterilizing virus-containing solutions, as well as new uses as wave guides, molecular wires, optical triggers, and in molecular lasers, optical amplifiers, dye lasers, polymer grid triodes, light emitting and light responsive diode systems, LEDs, photovaltaics, as well as articles comprising an organic laser, and using the invention in methods and devices for in vivo diagnosis detecting IR emission by agents bound to body organs. Representative uses are disclosed by, for example, the following patents, which are incorporated herein by reference: U.S. Pat. No. 5,657,156 (van Veegel, et al.); U.S. Pat. No. 5,237,582 (Moses); U.S. Pat. No. 5,504,323 (Heeger, et al.); U.S. Pat. No. 5,563,424 (Yang, et al.); U.S. Pat. No. 5,062,428 (Chance); U.S. Pat. No. 5,859,251 (Reinhardt et al.); U.S. Pat. No. 5,770,737 (Reinhadt et al.); U.S. Pat. No. 5,062,428 (Chance); U.S. Pat. No. 5,881,089 (Berggren et al.); U.S. Pat. No. 4,895,682 (Ellis, et al.); U.S. Pat. No. 4,986,256 (Cohen); U.S. Pat. No. 4,668,670 (Rideout, et al.); U.S. Pat. No. 3,897,255 (Erickson); U.S. Pat. No. 3,899,334 (Erickson); U.S. Pat. No. 3,687,863 (Wacher); U.S. Pat. No. 4,647,478 (Formanek, et al.); and U.S. Pat. No. 4,957,615 (Ushizawa, et al.). Further uses are disclosed are disclosed by, for example, U.K. Patent Application 2,225,963 (Casson, et al.); International Application WO 89/11277 (Dixon, et al.); International Application WO 91/09631 (Matthews, et al.); International Application WO 98/50989 (Forrest et al.); International Application WO 01/49475 (Peumans et al.); European Patent Application 85105490.8 (Weishaupt, et al.); European Patent Application 90202953.7 (Terrell, et al.); European Patent Application 89304234.1 (Matsushima, et al.); Lehn, Angew. Chem. Int. Ed. Engl., 1988, 27, 89; Wasielewski, Chem. Rev., 1992, 92, 435; Mansury, et al., J. Chem. Soc., Chem. Comm., 1985, 155; Groves, et al., J. Am. Chem. Soc., 1983, 105, 5791; and Giroud-Godquin, et al., Angew. Chem. Int. Ed. Engl., 1991, 30, 375. It is believed that the porphyrins of the invention can be substituted for the porphyrins disclosed in each of the foregoing publications.

A flurophore according to the invention is an emissive compound in which the spin multiplicity of the two states involved in the radiative transition (typically an electronically excited state and the ground state) have identical spin multiplicities. A lumophore is an emissive compound in which one of the two states involved in the radiative transition (typically the electronically excited state) derives from substantial mixing of two or more orbital configurations having different spin multiplicities [see for example, Lakowicz, J. R. Principles of Fluorescence Spectroscopy (Plenum Press, New York, 1983); Turro, N. J. Modern Molecular Photochemistry (The Benjamin/Cummings Publishing Co., Inc., Menlo Park, 1978)]. A phosphore according to the invention is an emissive compound in which the spin multiplicity of the two states involved in the radiative transition (typically an electronically excited state and the ground state) differ in their respective spin multiplicities. A laser dye according to the invention is any organic, inorganic, or coordination compound that has been established previously to lase. Representative laser dyes can be found in Birge, R. R.; Duarte, F. J. Kodak Optical Products, Kodak Publication JJ-169B (Kodak Laboratory Chemicals; Rochester, N.Y. (1990). Representative laser dyes include:

| | |
|---|---|
| p-terphenyl | Sulforhodamine B |
| p-quaterphenyl | Rhodamine 101 |
| carbostyryl 124 | Cresy Violet perchiorate |
| popop | DODC Iodide |
| Coumarin 120 | Sulforhodamine 101 |
| Coumarin 2 | Oxazine 4-perchiorate |
| Coumarin 339 | DCM |
| Coumarin 1 | Oxazine 170 perchlorate |
| Coumarin 138 | Nile Blue A Perchlorate |
| Coumarin 106 | Oxatine 1 Perchlorate |
| Coumarin 102 | Pyridine 1 |
| Coumarin 314T | Styryl 7 |
| Coumarin 338 | HIDC Iodide |
| Coumarin 151 | DTPC Iodide |
| Coumarin 4 | Cryptocyanine |
| Coumarin 314 | DOTC Iodide |
| Coumarin 30 | HITC Iodide |
| Coumarin 500 | HITC Perchiorate |
| Coumarin 307 | DTTC Iodide |
| Coumarin 334 | DTTC Perchiorate |
| Coumarin 7 | IR-144 |
| Coumarin 343 | HDITC Perchiorate |
| Coumarin 337 | IR-140 |
| Coumarin 6 | IR-132 |
| Coumarin 152 | IR-125 |
| Coumarin 153 | Boron-dipyrromethene |
| HPTS | Flourescein |
| Rhodamine 110 | 2,7-dichlorofluorescein |
| Rhodamine 6G | Rhodamin 19 Perchlorate |
| Rhodamine B | |

In preferred embodiments, the electronic structure of the component moieties in compounds of the invention are similar. The respective one-electron oxidation and reduction potentials thereof preferably differ by less than 250 mV. The energies of the respective lowest energy electronic transitions preferably differ by less than 2500 cm⁻¹.

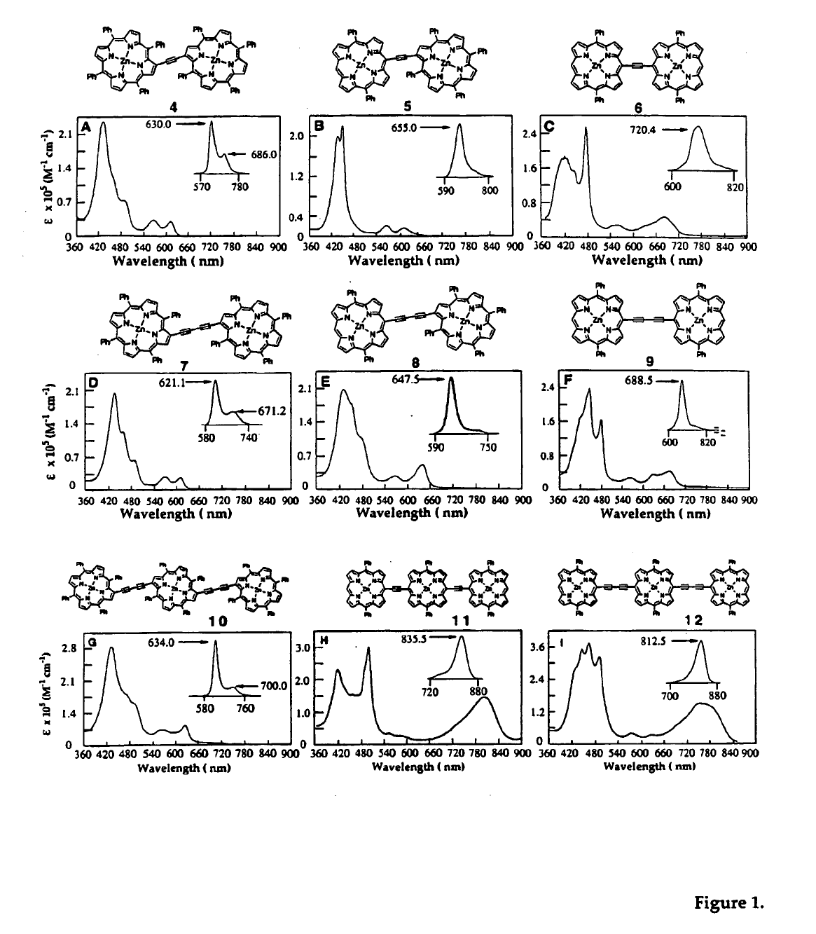

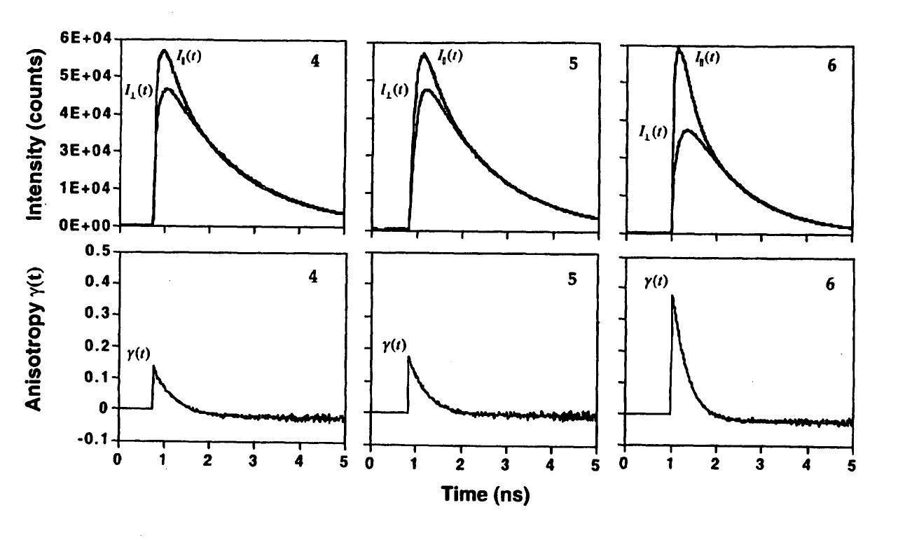

It has been found in accordance with the present invention that a wide variety novel highly conjugated porphyrin-based chromophore systems of the invention have unusual electooptic properties, and can function as collective oscillators. The formation of a collective oscillator and cooperative emission requires coupling of the transition dipoles of monomeric pigments. The compounds in the preferred embodiment of the invention are a class of multichromophoric systems that display extremely strong pigment-pigment electronic coupling; these assemblies feature ethyne and butadiyne moieties that directly link the carbon frameworks of their constituent porphyrin building blocks (FIG. 1). These ethyne- and butadiyne-bridged porphyrin arrays exhibit a number of surprising and unexpected optoelectronic characteristics, and are remarkable in that their optical absorption profiles, emission wavelengths, redox properties, as well as spin distribution and orientation in their photoactivated triplet states, are regulated extensively by the mode of porphyrin-to-porphyrin linkage topology. The ability to modulate comprehensively such a broad range of photophysical properties stems from flexible synthetic methodology that permits the extent of ground- and excited-state electronic coupling between pigments in these systems to be varied over a wide range. Notably, because the magnitude of pigment-pigment electronic interactions in these supramolecular assemblies is large relative to the vibronic modes that typically broaden electronic transitions irrespective of the nature of chromophore-chromophore connectivity, molecular structure regulates intimately the nature of pigment transition dipolar interactions and the corresponding photophysics of their respective electronically excited singlet states.

In preferred embodiments, the compounds of the invention are synthetic multichromophoric systems that exhibit one or more of the following optical properties: (i) low energy emission excited states in which the transition dipoles of the constituent pigment building blocks are correlated in defined phase relationships, (ii) excited state polarization over long timescales, (iii) emission quantum yields that have an unusual dependence upon supramolecular structure and emission wavelength, (iv) the hallmarks of collective oscillator behavior in their respective electronically-excited states, and (v) extreme superradiance, the magnitude of which exceeds the maximal value predicted classically (eq 1). Integrated emission oscillator strengths that are large with respect to that manifest by the benchmark monomeric chromophore.

In another aspect of the invention, the multichromophoric systems are generated by the process of providing a conjugated compound comprising at least two covalently bound moieties and exposing the conjugated compound to an energy source for a time and under conditions effective to cause the compound to emit light. The light emitted is preferably in the range of 650-2000 nm. The moieties used are, for example, porphyrins, and they may be bound by at least one carbon-carbon double bond, carbon-carbon triple bond, or a combination thereof. The bond can be, for example, ethynyl, ethenyl, allenyl, or butadiynyl. In another aspect of the invention, the moities may, for example, be bound by a combination of those units, or at least one imine, phenylene, or thiophene group.

Time-Resolved Fluorescence Spectroscopy

In the present invention, the isotropic and anisotropic dynamics of the lowest energy singlet excited (S₁) states of benchmark ethyne-elaborated (porphinato)zinc(II) monomers (5-ethynyl-10,20-diphenylporphinato)zinc(II) (Compound 1), (5,15-diethynyl-10,20-diphenylporphinato)zinc(II) (Compound 2), and (2-ethynyl-5,10,15,20-tetraphenylporphinato)zinc(II) (3) as well as those of conjugated (porphinato)zinc(II) arrays (Compounds 4-12) (FIG. 1) were characterized employing the time-correlated single-photon counting (TCSPC) spectroscopic technique.

The fluorescence anisotropy (r(t)) measured at time t following optical excitation is obtained from the parallel (I_{∥}) and perpendicular (I_{⊥}) transient signals using the following expression:

$$r ( t ) = \frac{{I}_{∥} ( t ) - {I}_{⊥} ( t )}{{I}_{∥} ( t ) + 2 {I}_{⊥} ( t )} \qquad ( 2 )$$

The magnitude of the initial anisotropy, r(₀), depends on the respective degeneracies and polarizations of the absorptive and emissive states. The value of r₍₀₎ in a dilute solution (eq 3) is a product of the angular displacement (α) of the absorption and emission dipoles and the loss of anisotropy due to photoselection (2/5).

$${r}_{( 0 )} = \frac{2}{5} ( \frac{3 {cos}^{2} α - 1}{2} ) \qquad ( 3 )$$

In the absence of coherence effects, initial fluorescence anisotropies for chromophore systems based on (porphinato)metal species will fall into four limiting cases: (i) if the excited-state degeneracy is not broken, the initial excited state population will randomize between orthogonal and energetically equivalent x-and y-polarized S₁ states, giving rise to a r₍₀₎ value of 0.1; (ii) when excited-state degeneracy is removed and the absorption and emission dipoles are parallel (α=0), r₍₀₎ will equal 0.4; (iii) when excited-state is singly degenerate and the absorption and emission dipoles are orthogonal (α=90°), a r₍₀₎ value of −0.2 will be observed; and (iv) if overlapping (multiple) states of different polarizations and degeneracies are either pumped or probed, r₍₀₎ values intermediate between the −0.2 and 0.4 extremes will be manifest. It is important to note that, for the present invention, the actual measured value of r₍₀₎ depends intimately upon the experimental timescale; for example, if the interrogated absorption and emission dipoles are parallel, but an experimentally determined value of r₍₀₎ less than 0.4 is manifest, it generally indicates the existence of relaxation processes that occur on time scales shorter than the time resolution of the experiment. Such processes can involve nuclear dynamics (e.g., rotational or librational motion) of the molecule, or electronic (vibronic) relaxation pathways. Hence, in the present invention the degree of energetic splitting between orthogonal excited states, as well as chromophore size and shape, will determine the timescales over which the fluorescence anisotropy decays to zero (r(t)=0) and whether or not the experimental time domain is adequate to measure initial anisotropy values at the 0.4 and −0.2 extrema.

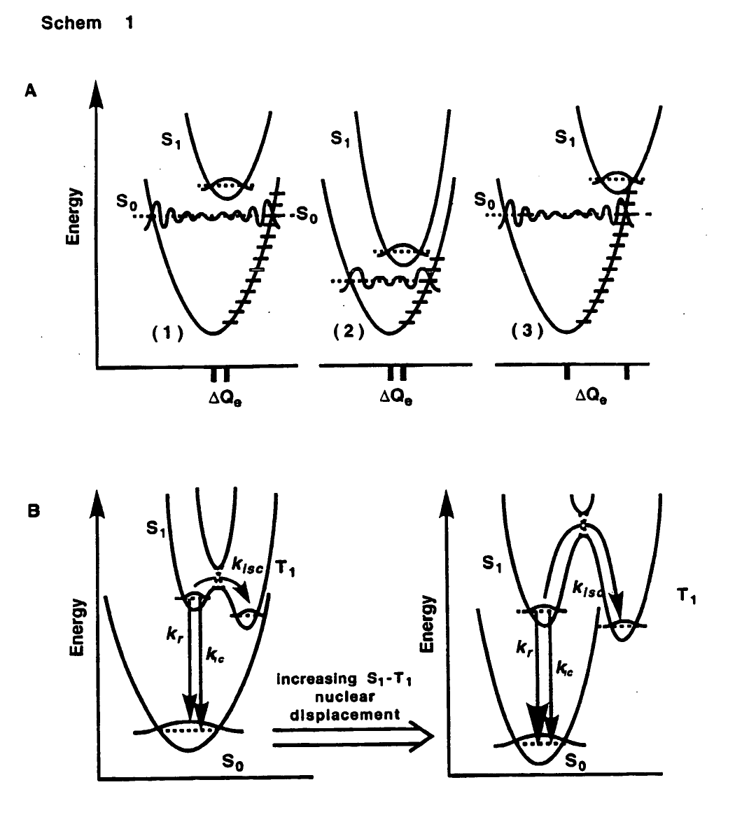

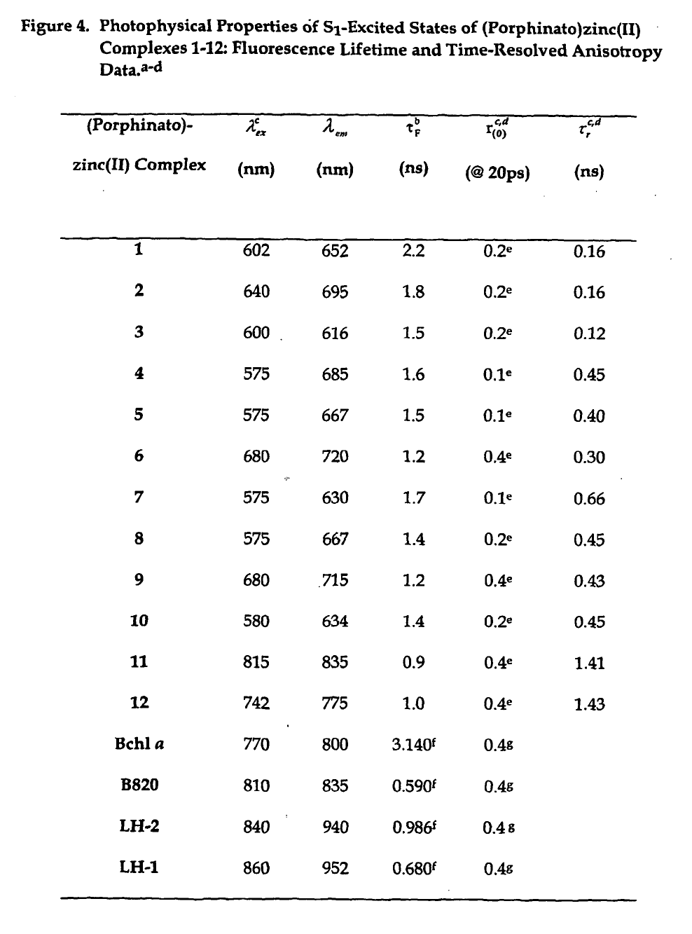

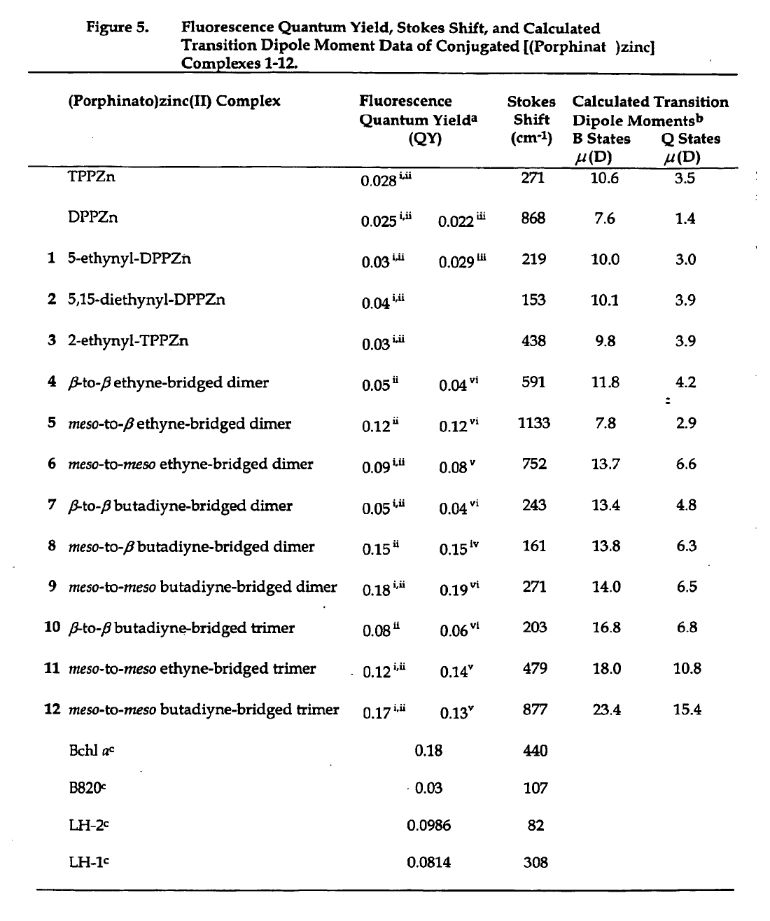

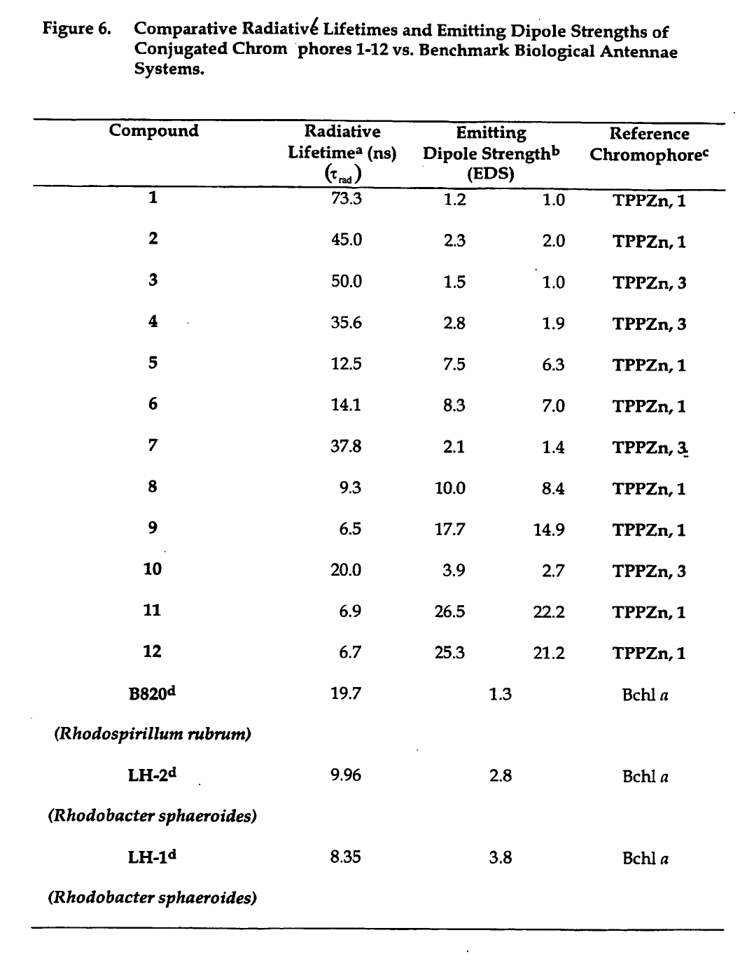

S₁-excited state lifetime and time-resolved fluorescence anisotropy data for the present invention are summarized in FIG. 4 for compounds 1-12; typical isotropic (magic angle) and anisotropic fluorescence decay profiles for these species are presented in FIG. 2. In these experiments, samples were excited on the low energy side of their respective lowest energy Q absorption bands; fluorescence decays were probed at wavelengths to the red of the emission λₘₐₓ. Monoexponential decay of the isotropic fluorescence was observed (FIG. 4); notably, all of these species of the present invention possess similar (ns) fluorescence lifetimes (τ_{F}). In contrast, the anisotropic fluorescence dynamics vary extensively in compounds 1-12 of the present invention. 5,10,15,20-Tetraphenylporphinato)zinc(II) (TPPZn), a porphyrinic photophysical benchmark, possesses a doubly degenerate S₁ excited state and displays an initial anisotropy (r₍₀₎=0.1; t=20 ps) following electronic excitation on the red edge of the lowest energy Q transition. The measured values of the initial anisotropy (r₍₀₎=0.2; t=20 ps) for compounds 1, 2, and 3 of the present invention show that expansion of porphyrin conjugation via meso-ethynyl moieties introduces an electronic perturbation sufficient to cause a splitting of the x- and y-polarized transitions.

Compounds 4-12 of the present invention express fluorescence anisotropies measured 20 ps after excitation that range from 0.1 to 0.4, indicating that chromophoric excited states can be prepared that range from doubly degenerate and nonpolarized (r₍₀₎=0.1), to singly degenerate and highly polarized (r₍₀₎=0.4). The nature of the porphyrin-to-porphyrin linkage topology is clearly important in establishing the magnitude of the initial anisotropy. Note that β-to-β bridged chromophores display r₀ values of ˜0.1 (compounds 4, 7, and 10), while only meso-to-meso bridged (porphinato)zinc(II) chromophores (compounds 6, 9, 11, and 12) exhibit values of 0.4. In addition to the topologically dependent magnitude of pigment-pigment electronic coupling, the extent of conformational mobility about the conjugated bridge likely plays a role in determining the magnitude of r₍₀₎ in meso-to-β bridged pigments (compounds 5 and 8).

Time-resolved experimental data show that the initial anisotropies for compounds 4-12 of the present invention decay typically via single exponential processes; these results indicate that the fluorescence anisotropy at time t after excitation is determined by the magnitude of the rotational diffusional time constant (τᵣ), [r(t)=r₀e^{(-t/τ}^{ᵣ}⁾]. These data evidence that fast electronic dephasing processes are absent, and that the phase relationship of the individual pigment dipoles remain correlated throughout the entire lifetime of the S₁-excited state in compounds 4-12; this is seen most dramatically in meso-to-meso ethyne- and butadiyne-bridged compounds 6, 9, 11, and 12, which manifest emissive, singly degenerate S₁ states polarized exclusively along their respective highly conjugated axes.

The fact that, in the preferred embodiment, ethyne- and butadiyne-bridged porphyrin arrays 4-12 display fluorescence lifetimes (0.9≦τ_{F}≦1.7 ns) similar in magnitude to that exhibited by their respective conjugated, monomeric building blocks 1-3 (1.5<τ_{F}≦2.2 ns), underscores the unusually long timescales over which excited state polarization can be maintained. Note that the emission maxima of compounds 4-12 span a 4,000 cm⁻¹ energy domain (621-836 nm; FIG. 1), indicating that both fluorescence wavelength and excited-state anisotropy can be highly modulated in these systems without significant diminution of τ_{F}.

Superradiant Emissi n

FIG. 5 chronicles the Stokes shifts, B- and Q-state transition dipole moments, and fluorescence quantum yields (QYs) for compounds 1-12. Note that emission QYs of ethyne-elaborated monomeric porphyrin compounds 1-3 exceed that measured for TPPZn and (5,15-diphenylporphinato)zinc(II) (DPPZn) benchmarks. Congruently, QYs determined for bis- and tris[(porphinato)zinc(II)] compounds 4-12 are larger than that measured for the monomers 1-3; note also that for both bis-(compounds 4-9) and tris[(porphinato)zinc(II)] (compounds 10-12) species, the absolute magnitudes of the QYs vary with linkage topology and the length of the cylindrically π-symmetric bridge that connects the aromatic macrocycles. When analyzed in context of the dynamical data presented in FIG. 4, the results summarized in FIG. 5 lead to a number of startling conclusions.

It has been noted that when the excited-state energy is modulated in a series of compounds based on a single emissive chromophore, the observed radiationless decay rate constant (kₙᵣ) for a specific pigment should follow a predictable dependence upon the respective degree of vibrational overlap between the relevant ground and excited states. This quantum effect is commonly referred to as the energy gap law; FIG. 3A, highlights its dependence upon extent of initial and final state energy separation, and the magnitude of equilibrium nuclear displacement (ΔQ) between these electronic states. As shown in FIG. 3A potential energy diagrams 1 and 2, decreasing the S₀−S₁ energy gap leads to enhanced vibrational wavefinction overlap, which effects a corresponding increase in kₙᵣ. Because τ_{F} is equal to the inverse sum of the radiative (kᵣ) and nonradiative rate constants (τ_{F}=(kᵣ+kₙᵣ)⁻¹), excited state lifetimes diminish correspondingly with decreasing emission energies. This simple prediction has now been verified in a number of pigment systems.

When a significant deviation from the expected linear dependence of ln(kₙᵣ) upon emission energy is observed within a series of related chromophores, it typically indicates that equilibrium ground- and excited-state nuclear displacements are not uniform within these pigments. Relevant to this study, it is important to point out that progressive expansion of chromophore conjugation has been established as a facile means to introduce such electronic structural perturbations. This is shown in FIG. 3A potential energy diagrams 1 and 3; note as ΔQ decreases at a constant S₀−S₁ energy separation, vibrational overlap decreases thus diminishing the magnitude of kₙᵣ. This effect is well documented in classic work by Meyer, which shows that augmentation of π electronic delocalization in ruthenium polypyridyl complexes results in substantially diminished kₙᵣ values and enhanced emission lifetimes relative to the ruthenium tris(bipyridyl) archetype; such an approach provides a viable strategy to engineer long-lived pigment excited states that possess emission energies that are reduced relative to the parent chromophore.

It is crucial to note, however, that the fabrication of red-emitting chromophores possessing long excited-state lifetimes via such an energy-gap-law-based a strategy does not come without a price. Because the size of the radiative rate constant kᵣ decreases substantially within a given class of isolated chromophores as the optical band gap narrows (eq 1), the magnitude of the emission quantum yield

$$( QY ; QY = \frac{{k}_{r}}{( {k}_{r} + {k}_{n r} )} )$$

falls dramatically. Engineering even modest shifts of emission energy (on the order of ˜2000 cm⁻¹) through chromophore conjugation expansion, has been shown to effect greater than ten-fold reductions in the observed emission QY with respect to that of the original pigment complex.

Taken in context of this discussion of the energy gap law, compounds 4-12 of the present invention are spectacular in that they exhibit both long fluorescence lifetimes and emission quantum yields that exceed significantly that of their constituent (porphinato)zinc(II) building blocks; importantly, the fluorescence QYs for compounds 4-12 are substantially augmented relative to simple (porphinato)zinc(II) complexes, despite the fact that the λₑₘ maxima for these species reside 700 to 4600 cm⁻¹ lower in energy than that for the TPPZn reference chromophore (FIGS. 4-6). Because the radiative transition probability is proportional to the cube of the emission energy (eq 1), in order for compounds 4-12 of the present invention to feature simultaneously substantial fluorescence lifetimes and emission quantum yields relative to their monomeric (porphinato)zinc(II) benchmarks, these multichromophoric systems must be behaving as collective oscillators(<μ>₄₋₁₂>><μ>_{TPPZn}) (eq 1).

The extent to which a pigment aggregate is superradiant is generally expressed in terms of a superradiance enhancement factor (emitting dipole strength) in which the experimentally determined <μ>_{aggregate} value is reference against <μ> measured for an appropriate monomeric pigment benchmark. The superradiance enhancement factor is thus a direct observable that is often taken as a classical measure of the exciton diffusion length.

Emitting dipole strengths (EDSs) and radiative lifetimes (τ_{rad}) for compounds 1-12 are listed in FIG. 6. While these data show that, in the present invention, bis-and tris(pigment) arrays 4-12 all manifest dramatic superradiance enhancement factors, the magnitudes of the EDSs determined for oligo[(porphinato)zinc(II)] systems of the present invention featuring meso-to-meso or meso-to-β linkage topologies (compounds 5, 6, 8, 9, 11, and 12) are particularly striking: they greatly exceed the expected maximal values (i.e., 2 and 3) predicted for ensembles composed of two and three respective monomeric pigment units (eq 1).

EDS values of this magnitude for similarly sized conjugated oligomers are without precedent. Likewise, superradiant conjugated polymers fabricated to date have exploited monomer units with transition dipole moments considerably smaller than that manifest by porphyryl moieties. Given the lack of appropriate photophysical benchmarks among superradiant conjugated organic materials, it is useful to compare these data to those obtained for the superradiant biological light harvesting proteins, which feature strongly-coupled chromophore ensembles composed of similar pigment monomeric units (bacteriochlorophylls and chlorophylls). Analogous data obtained for the biological benchmarks are shown for comparison in FIGS. 4-6. Using the EDS of the bacteriochlorophyll a (Bchl a) monomer as a chromophoric reference, and analyzing appropriate photophysical data obtained for the B820 subunit of the LH-2 protein of Rhodospirillum rubrum and the intact light harvesting complexes LH-1 and LH-2 of Rhodobacter sphaeroides in terms of the Einstein relation (eq 1), van Grondelle has shown that the superradiance enhancement factors for these biological pigment-protein complexes are respectively 1.3, 2.8, and 3.8. These results implied that the exciton diffusion lengths in the long-wavelength absorbing pigment assemblies of B820, LH-1, and LH-2 corresponded to distances defined by these respective numbers of Bchl a units; because the strongly coupled chromophore arrays of B820, LH-1, and LH-2 possess respectively 2, 16, and 32 pigments, these experiments suggested that while the extent of excited-state delocalization is significant in these LHCs, it is not global in nature.

Clearly, the EDSs determined ethyne- and butadiyne-bridged (porphinato)zinc(II) arrays that feature meso-to-meso or meso-to-β linkage motifs must arise from factors supplemental to the collective, in-phase oscillation of the individual pigment dipoles in these conjugated chromophore systems. These EDSs can be rationalized considering the established triplet photophysics of these species. In contrast to the singlet excited states of compounds 6, 8, 9, 11, and 12, which evince substantial delocalization of electron density, photoexcited EPR spectroscopic studies establish conclusively that the T₁-excited-state electron density distributions in compounds 4-12 of the present invention are all highly localized. Importantly, these experiments show that the spatial extent of T₁-state wavefunction in these species in no case exceeds the dimensions defined by a single (porphinato)zinc(II) unit and its pendant, cylindrically π-symmetric (ethyne or butadiyne) substituents. The genesis of this T₁ wavefunction localization in compounds 4-12 may derive from large lattice relaxations, which are known to diminish the spatial extent of triplet electronic states relative to excited Sₙ states in oligophenylene ethynylenes, or from fundamental electronic differences between the singlet and triplet excitation manifolds that can be rationalized within the context of the point-dipole approximation of the general exciton model.

Because the exciton resonance scales with the square of the transition moment, it is likely that high oscillator strength absorbers (compounds 4-12) possess unusually large Franck-Condon barriers to S₁−T₁ intersystem crossing. Moreover, as S₁ excite-state electronic delocalization increases in this series (λₑₘ increases), these Franck-Condon barriers would be expected to increase progressively as well FIG. 3B, since similar, highly localized T₁ states are manifest for a given porphyrin-to-porphyrin linkage topology regardless of the size of the pigment array. Because τ_{F}=(kᵣ+kₙᵣ)⁻¹, and the magnitude of the nonradiative decay rate constant kₙᵣ corresponds to the sum of all kinetic processes that non-emissively quench the S₁-excited state (kₙᵣ=k_{IC}+k_{ISC}, where k_{ISC}=S₁−T₁ intersystem crossing rate constant and k_{IC} represents the overall rate constant for the S₁−S₀ non radiative internal conversion process), as k_{ISC}→0, the magnitude of the fluorescence lifetime correspondingly increases FIG. 3B. As noted above, the energy gap law predicts that decreased S₀−S₁ energy separations will lead to increased values of k_{IC} within a given class of chromophores; while this undoubtedly plays a role in the S₁ photophysics for compounds 4-12, at least over the emission energies spanned by these species, augmentation of the magnitude of k_{IC} is apparently more than compensated by the corresponding diminution of k_{ISC} with increasing λₑₘ. This effect causes the magnitude of the fluorescence lifetime to remain relatively constant throughout compounds 1-12, and is a primary determinant for the unusually large fluorescence QYs observed for the red-emitting chromophores in this series. Thus, in addition to in-phase oscillation of strongly coupled pigment transition dipoles, concomitant reduction of k_{ISC} with increased S₁-state electronic delocalization for these species plays a key role in establishing the extreme superradiant behavior highlighted in FIG. 6.

This work shows that excited-state deactivation pathways that dominate the photophysics of monomeric pigments need not necessarily control the excited-state dynamics of their corresponding strongly-coupled chromophore assemblies; hence the supermolecular multipigment systems of the present invention that exist as distinct photophysical entities can be constructed from simple chromophoric building blocks. These ethyne- and butadiyne-bridged (porphinato)zinc(II) assemblies show the essential characteristics of the pigment assemblies of the biological light harvesting proteins: substantial pigment-pigment coupling, high excited-state polarization, and coupled oscillator photophysics. When such conjugated assemblies are engineered to possess singly degenerate excited states, high and low frequency vibrational modes of the chromophore and solvent do not significantly impact excited-state electronic dephasing, and the polarized, dipole-dipole correlated nature of these singlet excited states is maintained over long, ns timescales.

Analysis of the fluorescence intrinsic decay rate and quantum yield data show that in the present invention the ethyne- and butadiyne-bridged multiporphyrin species that manifest high excited-state anisotropies display extreme superradiance enhancement factors: such photophysics derive from the fact that these conjugated pigment arrays behave as collective oscillators, and feature large Frank-Condon barriers for intersystem crossing between their respective S₁ and T₁ states. These results indicate that substantial emitting dipole strengths can in fact be realized for low energy fluorescing materials, and that classic energy gap law considerations that place important restrictions upon the elaboration of isolated pigments that manifest high quantum yield, low energy fluorescence, do not preclude the design of supermolecular systems that manifest such photophysics.

Finally, this work suggests that the combination of monomer units having large absorption oscillator strengths, with monomer-to-monomer linkage motifs that assure strong coupling, dipole-dipole alignment, and large values of the excited-state anisotropy, may constitute a general strategy for the fabrication of electrooptic materials that exhibit extreme superradiance. Because low energy optical band gaps, high anisotropy singlet excited-states, and extreme superradiance can be engineered in parallel, the design concepts articulated herein bear relevance to the fabrication of photonic materials, and device applications where pigment organization, divergent cross sections for singlet and triplet exciton formation from injected charge carriers, and large optical gain, are held at a premium.

Additional objects, advantages, and novel features of this invention will become apparent to those skilled in the art upon examination of the following examples thereof, which are not intended to be limiting. Additional synthetic techniques for compounds of the invention are disclosed in: (1) Highly-Conjugated, Acetylenyl-Bridged Porphyrins: New Models for Light-Harvesting Antenna Systems, V. S.-Y. Lin, S. G. DiMagno, and M. J. Therien, S science (Washington, D.C.) 1994, 264, 1105-1111; (2) The Role of Porphyrin-to-Porphyrin Linkage Topology in the Extensive Modulation of the Absorptive and Emissive Properties of a Series of Ethynyl- and Butadiynyl-Bridged Bis- and Tris(porphinato)zinc Chromophores, V. S.-Y. Lin and M. J. Therien, Chem. Eur. J. 1995, 1,645-651; (3) Singlet and Triplet Excited States of Emissive, Conjugated Bis(porphyrin) Compounds Probed by Optical and EPR Spectroscopic Methods, R. Shediac, M. H. B. Gray, H. T. Uyeda, R. C. Johnson, J. T. Hupp, P. J. Angiolillo, and M. J. Therien, J. Am. Chem. Soc. 2000,122, 7017-7033.

### EXAMPLE 1

### 5,15-Diphenylporphyrin

A flame-dried 1000 ml flask equipped with a magnetic stirring bar was charged with 2,2-dipyrrylmethane (458 mg, 3.1 mmol), benzaldehyde (315 μl, 3.1 mmol), and 600 ml of freshly distilled (CaH₂) methylene chloride. The solution was degassed with a stream of dry nitrogen for 10 minutes. Trifluoroacetic acid (150 μl, 1.95 mmol) was added via syringe, the flask was shielded from light with aluminum foil, and the solution was stirred for two hours at room temperature. The reaction was quenched by the addition of 2,3-dichloro-5,6-dicyano-1,4-benzoquinone (DDQ, 900 mg, 3.96 mmol) and the reaction was stirred for an additional 30 minutes. The reaction mixture was neutralized with 3 ml of triethylamine and poured directly onto a silica gel column (20×2 cm) packed in hexane. The product was eluted in 700 ml of solvent. The solvent was evaporated, leaving purple crystals (518 mg., 1.12 mmol, 72.2%). This product was sufficiently pure for further reactions. Vis(CHCl₃): 421 (5.55), 489 (3.63), 521 (4.20), 556 (4.04), 601 (3.71), 658 (3.73).

### EXAMPLE 2

### 5,15-Dibromo-10,20-Diphenylporphyrin

5,15-Diphenylporphyrin (518 mg, 1.12 mmol) was dissolved in 250 ml of chloroform and cooled to 0° C. Pyridine (0.5 ml) was added to act as an acid scavenger. N-Bromosuccinimide (400 mg, 2.2 mmol) was added directly to the flask and the mixture was followed by TFLC (50% CH₂Cl₂/hexanes eluant). After 10 minutes the reaction reached completion and was quenched with 20 ml of acetone. The solvents were evaporated and the product was washed with several portions of methanol and pumped dry to yield 587 mg (0.94 mmol, 85%) of reddish-purple solid. The compound was sufficiently pure to use in the next reaction. Vis(CHCl₃): 421 (5.55), 489 (3.63), 521 (4.20), 556 (4.04), 601 (3.71), 658 (3.73).

### EXAMPLE 3

### 5,15-Dibromo-10,20-Diphenylporphyrinato Zinc

5,15-Dibromo-10,20-diphenylporphyrin (587 mg, 0.94 mmol) was suspended in 30 ml DMF containing 500 mg ZnCl₂. The mixture was heated at reflux for 2 hours and poured into distilled water. The precipitated purple solid was filtered through a fine fritted disk and washed with water, methanol, and acetone and dried in vacuo to yield 610 mg (0.89 mmol, 95%) of reddish purple solid. The compound was recrystallized from THF/heptane to yield large purple crystals of the title compound (564 mg, 0.82 mmol, 88%). Vis(THF): 428 (5.50), 526 (3.53), 541 (3.66), 564 (4.17), 606 (3.95).

### EXAMPLE 4

### Meso-substituted Porphyrins

General Procedure

In each of the following examples, 5,15-Dibromo-10,20-diphenylporphyrinato zinc (0.1 mmol), and Pd(PPh3)4 (0.0025 mmol) were dissolved in 35 ml of distilled, degassed THF in a sealed storage tube with the 1 mmol of the indicated organometallic reagent and warmed at 60° C. for 48 hours. The reaction was monitored by TLC on withdrawn aliquots. The mixture was quenched with water, extracted with chloroform, dried over CaCl2, evaporated and purified by column chromatography.

A. 5,15-Diphenyl-10,20-dimethylporphyrinato zinc

The organometallic reagent was methyl zinc chloride prepared from methyl lithium and anhydrous zinc chloride in THF.

The crude solid was dissolved in THF/heptane, poured onto 10 g silica gel and evaporated to dryness. This silica gel was loaded onto a column packed in 50% CH₂Cl₂/hexane. A single band was eluted (50% CH₂Cl₂/hexane) to yield pure 5,15-diphenyl-10,20-dimethylporphyrinato zinc (48 mg, 88%). An analytical sample was recrystallized from THF/heptane by slow evaporation under N2. ¹H NMR (500 MHz, 3:1 CDCl₃, D₈-THF) epsilon 9.34 (d, 4H, J=4.6); 8.71 (d, 4H, J=4.6); 8.02 (dd, 4H, J1=7.5, J2=1.4); 7.57 (m, 6H); 4.51 (s, 6H). ¹³C NMR (125 MHz, 3:1 CDCl₃, Da-THF) epsilon 150.07 (0), 148.88 (0), 143.34 (0), 134.18 (1), 131.42(1), 128.09(1), 126.73(1), 125.88(1), 119.29(0), 113.74(0), 20.81(3). Vis (THF) 424 (5.58), 522 (3.40), 559 (4.12); 605 (3.88).

B. 5,15-Diphenyl-10,20-divinylporphyrinato zinc

The organometallic reagent was tri-n-butylvinyl tin.

The crude product was absorbed on silica and loaded onto a column packed in hexane. Elution was carried out with CH₂Cl₂(0-50%)/hexane. A small quantity of purple material led the main fraction. The main band was evaporated to yield pure 5,15-diphenyl-10,20-divinylporphyrinato zinc (53 mg, 91%). An analytical sample was recrystallized from chloroform. ¹H NMR (500 MHz, CDCl₃) epsilon 9.52 (d, 4H, J=4.7); 9.24(dd, 2H, J1=17.3, J2=9.1); 8.92 (d, 4H, J=4.7); 8.19 (dd, 4H, J1=6.8, J2=2.0); 7.75 (m, 6H); 6.48 (dd, 2H, J1=11.0, J2=1.9); 6.05 (dd, 2H, J1=17.3, J2=2.0). ¹³C NMR (125 MHz, CDCl₃) epsilon 163.40(1), 149.90(0), 149.21( 0), 142.83(0), 137.97(0), 134.40(1), 132.10(1), 130.39(1), 127.50(1), 126.73(2), 126.57(1), 121.05(0).

C. 5,15-Bis(2,5-dimethoxyphenyl)-10,20-diphenylporphyrinato zinc

The organometallic reagent was 2,5-dimethoxyphenyl lithium, prepared from 1,4-dimethoxybenzene and t-butyl lithium in ether at −78° C. The organolithium reagent was added to a solution of ZnCl₂ in THF to yield the organozinc chloride reagent. This reagent was used immediately.

At the completion of the reaction two highly fluorescent spots were visible by TLC. The crude product was chromatographed on silica using CHCl₃ as eluant. The first band off the column proved to be the C₂ₕ isomer of 5,15-bis(2,5-dimethoxyphenyl)-10,20-diphenylporphyrinato zinc. This band was evaporated leaving 33 mg (42%) of pure product. An analytical sample was recrystallized from chloroform. ¹H NMR (500 MHz,CDCl₃) epsilon 8.91 (s, 8H); 8.22 (d, 4H, J=6.5); 7.75 (m, 6H); 7.59 (d, 2H, J=2.2); 7.26 (broad s, 4H); 3.86 (s, H); 3.54 (s, 6H). ¹³C NMR (125 MHz, CDCl₃) epsilon 154.10(0), 152.30(0), 150.13(0), 143.00(0), 134.10(1), 132.62(0), 132.00(1), 131.44(1), 127.35(1), 126.44(1), 121.34(1), 120.69(0), 110.59(0), 114.76(1), 112.31(1), 56.70(3), 55.95(3). Vis-424 (5.64), 551 (4.34), 584 (3.43).

The C_{2V} isomer followed the C₂ₕ isomer off the column. The solvent was evaporated leaving 30 mg (32%) of pures, 15-bis(2,5-dimethoxyphenyl)-10,20-diphenylporphyrinato zinc. This compound is much more soluble in chloroform than the C₂ₕ isomer. The assignment of stereochemistry was made from the NMR data. ¹H NMR (500 MHz,CDCl₃) epsilon 8.90 (s, 8H); 8.21 (d, 2H, J=7.9); 8.19 (d, 2H, J=6.5); 7.73 (m, 6H); 7.58 (s, 2H); 7.24 (broad s, 4H); 3.84 (s, 6H); 3.53 (s, 6H). ¹³C NMR (125 MHz CDCl₃) epsilon 154.14 (0); 152.31 (0), 150.15 (0), 142.94(0), 134.40(1), 132.66(0), 132.02(1), 131.48(1), 127.37(1), 126.46(1), 126.44(1), 121.30(1), 120.72(0), 116.69(0), 114.73(1), 112.28(1), 56.75(3), 55.92(3).

D. 5,15-Bis[(4-methyl)-4′-methyl-2,2′-dipyridyl)]-10,20-diphenylporphyrinato zinc

The organometallic reagent was tri-n-butyl[(4-methyl)-4′-methyl-2,2′-dipyridyl)]tin, prepared by lithiating 4,4′-dimethyl-2,2′-dipyridyl with one equivalent of lithium diisopropylamide in THF at −78° C. and warming the reaction mixture to room temperature. The organolithium reagent was treated with 1.1 equivalent of tributyltin chloride. The resulting organotin reagent was used without further purification.

Chromatography of the crude reaction mixture was carried out on silica with a mixture of CH₂Cl₂, isopropanol, and triethylamine. The porphyrin was eluted in one broad band. The product obtained from this procedure (68% yield) was contaminated with a small amount (0.2 eq per eq of porphyrin) of triphenylphosphine. ¹H NMR (500 MHz, CDCl3) epsilon 9.37 (d, 4H, J=4.7); 8.87 (d, 4H, J=4.7); 8.52 (s, 2H); 8.29 (d, 2H, J=5.1); 8.20 (d, 2H, J=5.2); 8.10 (m, 6H); 7.71 (m, 6H); 7.01 (d, 2H, J=5.0); 6.88 (d, 2H, J=4.2); 6.46 (s, 4H); 2.32 (s, 6H).

E. 5,15-Bis(trimethylsilylethynyl)-10,20-diphenylporphyrinato zinc

The organometallic reagent was trimethylsilyl ethynyl zinc chloride prepared from trimethylsilylethynyl lithium and anhydrous zinc chloride in THF.

After 48 hours the reaction was bright green. The crude solid was absorbed on silica gel, loaded onto a column packed in hexane, and chromatographed with 20%-30% CH₂Cl₂/hexane. Clean separation of the product from the small quantities of deprotected products were obtained by this method. The solvents were evaporated and the purple solid was washed twice with and dried in vacuo. ¹H NMR (500 MHz, CDCl₃) epsilon 9.68 (d, 4H, J=4.6); 8.89 (d, 4H, J=4.6); 8.15 (dd, H, J1=7.9, J2=1.7); 7.75 (m, 6H); 0.58 (s, 18H). ¹³C NMR (125 MHz, CDCl₃) epsilon 152.22, 150.26, 142.10, 134.39, 132.77, 131.29, 27.69, 126,67, 115.08, 107.34, 102.00, 0.32.

### EXAMPLE 5

### Pyrrole-substituted Porphyrins

General Procedure

2-Bromo-5,10,15,20-tetraphenylporphyrinato zinc (0.1 retool) and palladium 1,1′-bis (diphenylphosphino) ferrocene) dichloride (Pd(dppf)Cl₂, 7 mg) were combined with 1.0 mmol of the organometal lic reagent indicated below in 35 ml dry, degassed THF. The solution was allowed to stand for 12 hours, the solvent evaporated, and the compound purified by flash chromatography.

A. 2-Vinyl-5,10,15,20-tetraphenyl porphyrinato zinc

The organometalic reagent was tributylvinyl tin.

The crude reaction mixture was chromatographed on silica and eluted with 50% CH₂Cl₂/hexane. ¹H NMR (250 MHz, CDCl₃) epsilon 8.97 (s, 1H); 8.90 (m, 4H); 8.87 (d, 1H, J=4.7); 8.79 (d, 1H, J=4.7); 8.20 (m, 6H); 8.06 (d, 2H, J=6.6); 7.74 (m, 12H); 6.39 (dd, 1H, J1=17.0, J2=9.1); 5.83 (dd, 1H, J1=17.1, J2=2.0); 5.01 (dd, 1H, J1=10.7, J2=2.0). Vis (CHCl₃) 426 (5.53), 517 (3.68); 555 (4.22), 595 (3.68).

B. 2-(2,5-dimethoxyphenyl)-5,10,15,20-tetraphenyl porphyrinato zinc

The organometallic reagent was 2,5-dimethoxyphenyl zinc chloride, prepared from the corresponding lithium reagent and anhydrous zinc chloride in THF/diethyl ether.

Flash chromatograph of the crude reaction mixture was carried out with chloroform. The title compound was isolated in 78% yield. ¹H NMR (500 MHz, CDCl₃) epsilon=8.94 (d, 1H, J=4.7); 8.93 (s,2H); 8.92 (d, 1H, J=4.8); 8.85 (s, 1H); 8.84 (d, 1H, J=4.7); 8.70 (d, 1H, J=4.7); 8.23 (m, 6H); 7.98 (d, 1H, J=7.0); 7.70 (m, 10H); 7.25 (quintet, 2H, J=7.4); 7.15 (t, 1H, J=7.0); 6.92 (d, 2H, J=3.1); 6.54 (dd, 1H, J1=9.0, J2=3.2); 6.40 (d, 1H, J=9.1); 3.68 (s, 3H); 3.42 (s, 3H). ¹³C NMR (125 MHz, CDCl₃) epsilon=152.59(0), 151.33(0), 150.46(0), 150.31(0), 150.27(0), 150.15(0), 150.12(0), 150.03(0), 148.30(0), 147.16(0), 143.32(0), 142.97(0), 142.86,140.71(0), 135.63(1), 135.20(1), 134.45(1), 134.13(0), 132.52(1), 132.02(1), 131.91(1), 131.82(1), 131.32(1), 129.27(0), 127.44(1), 127.38(1), 127.18(1), 126.53(1), 126.50(1), 124.91(1), 121.70(1), 122.36(0), 121.30(0), 120.91(0), 120.54(0), 113.15(1), 113.03(1), 110.35(1), 55.98(3), 54.87(3). Vis (CHCl3) 421.40(5.60), 513.2 (3.45), 549.75 (4.28), 587.15 (3.45).

C. 2-(Trimethylsilylethynyl)-5,10,15,20-tetraphenyl porphyrinato zinc

The organometallic reagent, trimethylsilylacetylide zinc chloride, was prepared from the corresponding lithium reagent and anhydrous zinc chloride in THF.

The crude reaction mixture was chromatographed on silica and eluted with 50% CH₂Cl₂/hexane. ¹H NMR (250 MHz, CDCl₃) epsilon 9.25 (s, 1H); 8.89 (m, 4H); 8.85 (d, 1H, J=4.9); 8.76 (d, 1H, J=4.9); 8.16 (m, 6H); 8.09 (d, 2H, J=7.1); 7.67 (m, 12H); 0.21 (s, 9H). Vis (CHCl3) 431 (5.43), 523 (shoulder); (4.18), 598 (3.67).

D. 2-n-butyl-5,10,15,20-tetraphenyl porphyrinato zinc

Butyl zinc chloride was prepared from n-butyllithium and anhydrous zinc chloride in THF.

The crude reaction mixture was chromatographed on silica and eluted with 50% CH₂Cl₂/hexane. ¹H NMR (250 MHz, CDCl₃) epsilon 8.97 (m, 4H); 8.91 (d, 1H, J=4.7); 8.77 (d, 1H, J=4.7); 8.74 (s, 1H); 8.22 (m, 6H); 8.13 (d, 2H, J=7.3); 7.77 (m, 12H); 2.81 (t, 2H, J=7.7); 1.83 (quint, 2H, J=7.8); 1.30 (quint, 2H, J=7.6); 0.91 (t, 3H, J=8.2).

### EXAMPLE 6

### Vinylic-bridged Porphyrins and Their Polymers

A. cis-Bis-1,2-[5-(10,15,20-triphenylporphyrinato) zinc]ethene

5-Bromo-10,15,20-triphenylporphyrinato zinc (0.2 mmol) and Pd(PPh3)4 (0.02 mmole) are dissolved in 25 ml dry, degassed THF. A solution of cis-bis(tri-n-butyltin)ethene (0.2 mmol) in 5 ml THF is added and the solution heated at reflux for 2 days. The reaction is quenched with water, extracted with methylene chloride, dried over calcium chloride, and the solvents are evaporated. The crude solid is chromatographed on silica using methylene chloride/hexane eluant to isolate a dimer having formula (3), wherein R_{A1}, R_{A3}, and R_{A4} are phenyl and M is Zn.

B. cis-Bis-1,2-[5-[10,15,20-tris(pentafluoro-phenyl)]-2,3,7,8,12,13,17,18-octakis-(trifluoromethyl) porphyrinato zinc]-1,2-difluoroethene

5-Bromo-10,15,20-tris(pentafluorophenyl)porphyrinato zinc (0.2 mmol) and Pd(PPh3)4 (0.02 mmol) are dissolved in 25 ml dry THF. A solution of cis-bis(tri-n-butyltin)-1,2-difluoroethene (0.2 mmol) in 5 ml THF is added and the solution heated at reflux for 2 days. The reaction is quenched with water, extracted with methylene chloride, dried over calcium chloride, and the solvents evaporated. The crude solid is chromatographed on silica using methylene chloride/hexanes eluent to isolate cis-bis-1,2-[5-[10,15,20-tris(pentafluorophenyl)porphyrinato zinc]-1,2-difluoroethene.

This material is dissolved in chloroform and reacted with a large excess of N-bromosuccinimide as in Example 2 to perbrominate positions R_{B1}—R_{B8} on both porphyrins. The resulting material filtered through a fine fritted disk and washed with water, methanol, and acetone, dried in vacuo, and then recrystallized from THF/heptane. cis-Bis-1,2-[5-[10,15,20-tris(pentafluoro-phenyl)-2,3,7,8,12,13,17,18-octabromoporphyrinato zinc is reacted with Pd(dppf) and a large excess of CuCF3 in the dark as in Example 4. After a reaction time of about 48 hours, the product is chromatographed on silica with CH₂Cl₂/CCl₄ eluent to yield the title compound.

C. Cofacial-bis-[cis-ethenyl meso-bridged]porphyrin[CEBP](Formula (5)) and Polymeric-bis-[cis-ethenyl meso-bridged]porphyrin [PABP](Formula (6))

5,15-Dibromo-10,20-diphenylporphyrinato zinc (0.2 mmol) and Pd(PPh₃)₄ (0.02 mmole) are dissolved in 25 ml dry, degassed THF. A solution of cis-bis(tri-n-butyltin)ethene (0.2 mmol) in 5 ml THF is added and the solution heated at reflux for 2 days. The reaction is quenched with water, extracted with methylene chloride, dried over calcium chloride, and the solvents are evaporated. The crude solid is chromatographed on silica using methylene chloride/hexane eluant to isolate the Cofacial-bis-[cis-ethenyl meso-bridged]zinc porphyrin complex of formula (5) and Polymeric-bis-[cisethenyl meso-bridged] porphyrin species of formula (6), wherein R_{A1} and R_{A3} are phenyl and M is Zn.

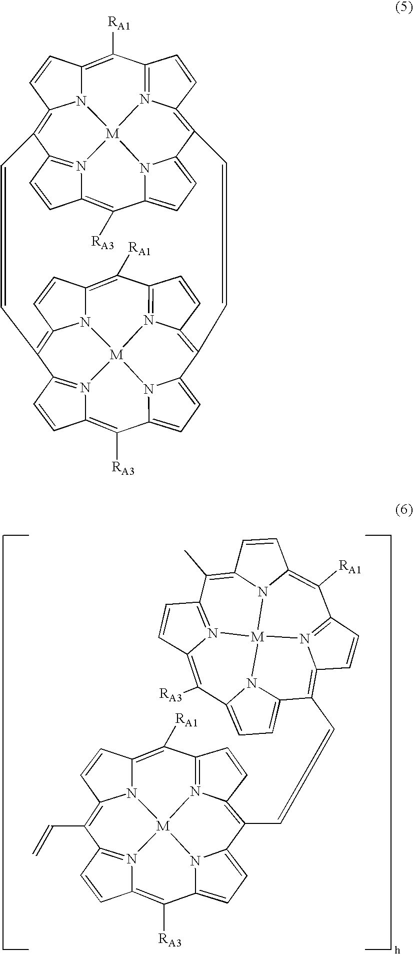

D. Fluorinated Cofacial-bis-[cis-ethenyl mesobridged]porphyrin[FCEBP]and Fluorinated Polymeric-bis-[cis-ethenyl meso-bridged]porphyrin [FPEBP]

5,15-Dibromo-10,20-bis(pentafluorophenyl) porphyrinato zinc (0.2 mmol) and Pd(PPh₃)₄ (0.02 mmol) are dissolved in 25 ml dry THF. A solution of cis-bis(tri-n-butyltin)-1,2-difluoroethene (0.02 mmol) in 5 ml THF is added and the solution heated at reflux for 2 days. The reaction is quenched with water, extracted with methylene chloride, dried over calcium chloride, and the solvents evaporated. The crude solid is chromatographed on silica using methylene chloride/hexanes eluent to isolate the Cofacial-bis-[cisethenyl meso-bridged] zinc porphyrin complex as well as the Polymeric-bis-[cis-ethenyl meso-bridged] porphyrin species. The cofacial and polymeric species are dissolved separately in chloroform. The cofacial porphyrin complex dissolved in chloroform and reacted with a large excess of N-bromosuccinimide as in Example 2 to perbrominate positions R_{B1}—R_{B8} on both porphyrins. The resulting material filtered through a fine fritted disk and washed with water, methanol, and acetone, dried in vacuo, and then recrystallized from THF/heptane to yield the title compound. The isolated material is reacted with Pd(dppf) and a large excess of CuCF₃ in the dark in a manner as in Example 4. After a reaction time of about 48 hours, the product is chromatographed on silica with CH₂Cl₂/CCl₄ eluent to yield a perfluorinated CEPB analogous to formula (5). Perfluorinated PEBP is synthesized in a similar manner, yielding a species analogous to formula (6) where highly fluorinated porphyrins are linked via fluorovinyl units.

E. Cofacial-bis-[1,8-anthracenyl-meso-bridged]porphyrin [CBAP] (Formula (7)) and Polymeric-bis-[1,8-anthracenyl-meso-bridged][PBAP]porphyrin (Formula (8)) 5,15-Dibromoporphyrinato zinc (0.2 mmol) and Pd(PPh₃)₄ (0.02 mmol) are dissolved in 25 ml dry, degassed THF. A solution of 1,8-anthracenyl-bis-(tributyl tin) (0.2 mmol) in 5 ml THF is added and the solution heated at reflux for 2 days. The reaction is quenched with water, extracted with methylene chloride, dried over calcium chloride, and the solvents are evaporated. The crude solid is chromatographed on silica using methylene chloride/hexane eluant to isolate the Cofacial-bis-[1,8-anthracenyl-meso-bridged]zinc porphyrin complex of formula (7) and the Polymeric-bis-[1,8-anthracenyl-meso-bridged]zinc porphyrin species of formula (8), where and R_{A1} and R_{A3} are phenyl and M is Zn.

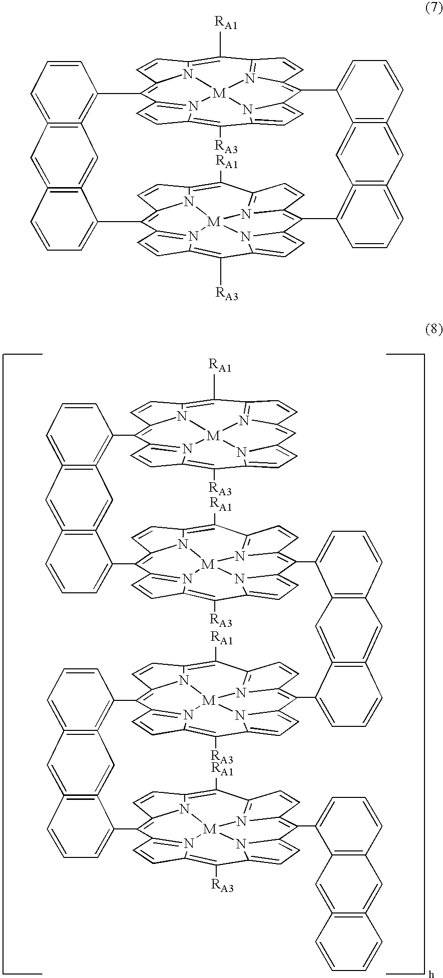

### EXAMPLE 7

### Acetylenic Porphyrin Polymers

A. Poly(5,15-bis(ethynyl)-10,20-diphenylporphyrinato zinc)

5,15-Bis(ethynyl)-10,20-diphenylporphyrinato zinc (0.2 mmol) in pyridin (20 ml) is slowly added to a solution of cupric acetate (0.4 mmol) in 20 ml 1:1 pyridine/methanol generally according to the procedure of Eglinton, et al., The Coupling of Acetylenic Compounds, p. 311 in Advances in Organic Chemistry, Raphael, et al., eds., 1963, Interscience Publishers.

B. Poly(5,15-bis(ethynylphenyl)-10,20-diphenylporphyrinato zinc)

5,15-Diethynyl-10,20-diphenylporphinato zinc (0.2 mmol) and 1,4-dibromobenzene are combined in a mixture of 30 ml toluene and 10 ml diisopropylamine. Cul (0.4 mmol) and Pd(Ph₃)₄ (0.02 mmol) are added and the mixture is heated at 65° C. for 3 days. The crude solid is washed with methanol and acetone and dried in vacuo.

Alternatively, the polymer is prepared from 1,4-diethynylbenzene and 5,15-dibromo-10,20-diphenylporphinato zinc via the identical procedure.

### EXAMPLE 8

### Doped Porphyrin Polymers

5,15-Bis(ethynyl)-10,20-diphenylporphyrinato zinc is polymerized according to the general procedure provided by Skotheim, ed., Handbook of Conducting Polymers, Volume 1, pp. 405-437, Marcel Dekker, 1986 using a catalytic amount of MoCl₅, Me(CO)₆, WCl₆, or W(CO)₅. The resultant polymer is then doped with an oxidant such as iodine or SbF₅.

### EXAMPLE 9

### Metalation of Brominated Porphyrins

Finely divided zinc metal was prepared generally according to the method of Rieke (J. Org. Chem. 1984, 49, 5280 and J. Org. Chem. 1988, 53, 4482) from sodium naphthalide and zinc chloride (0.18 mmol each) in THF. A solution of [5-bromo-10,20-diphenylporphinato]zinc (100 mg, 0.17 mmol) dissolved in 40 mL THF was added by syringe to the zinc metal suspension, and the mixture was stirred at room temperature overnight; during this time all of the zinc metal dissolved. The ring-metalated porphyrin is suitable for palladium-catalyzed coupling with a variety of aryl and vinyl halides.

### EXAMPLE 10

### Palladium-Catalyzed Cross-Coupling with Ring-Metalated Porphyrins

5-[(10,20-Diphenylporphinato)zinc]zinc bromide(0.2 mmol) is prepared in 15 mL of THF as in Example 9 above and is placed in a dry 100 mL Schlenk tube. A solution of 2-iodothiophene (0.4 mmol) in 5 mL of THF is added via syringe. Pd(dppf) (3 mg) is prepared by stirring a suspension of Pd(dppf)Cl₂ in THF over Mg turnings for 20 min. and is transferred into the reaction mixture by canula. The solution is stirred overnight, quenched with aqueous ammonium chloride, extracted with CH₂Cl₂, and dried over CaCl₂. The solvent is evaporated to dryness and chromatography is carried out with 1:1 CH₂Cl₂ as eluant. The product, [5-(2-thiophenyl)-10,20-diphenylporphinato]zinc, elutes in one band and is isolated in 90% yield.

### EXAMPLE 11

### Polymerization with Ring-Metalated Porphyrin Derivatives

[5,15-Bis(zinc bromide)-10,20-diphenylporphinato]-zinc (0.2 mmol) is prepared in 15 mL of THF as in Example 9 and is placed in a dry 100 mL Schlenk tube. A solution of [5,15-dibromo-10,20-diphenylporphinato]zinc (0.2 mmol) in 15 mL of THF is added by syringe. Pd(dppf) (3 mg) is prepared by stirring a suspension of Pd(dppf)Cl₂ in THF over Mg turnings for 20 min. and is transferred into the reaction mixture by canula. The mixture is heated at 60° C. for 3 days, cooled to room temperature and filtered through a fine-fritted glass disk. The filtered polymer is washed with hexane followed by methanol and dried in vacuo.

### EXAMPLE 12

### Carbonylation of [5-Bromo-10,20-Diphenylporphinato]Zinc

5-[(10,20-Diphenylporphinato)zinc]magnesium bromide (0.2 mmol) is prepared in 15 mL of THF as in Example 9 and is placed in a dry 100 mL Schlenk tube. The vessel is cooled to 0° C. and dry CO₂ gas is bubbled through the solution. The solution is stirred for 1 h at room temperature, quenched with 0.1 M HCl, extracted with CH₂Cl₂, and dried over CaCl₂. The solvent is evaporated to dryness and chromatography is carried out with THF:CH₂Cl₂ as eluant. Upon evaporation of the solvent [5-carboxy-10,20-diphenylporphinato]zinc is isolated in 85% yield.

### EXAMPLE 13

### Coupling on Unmetalated Porphyrin Derivatives

A. Using Organozinc Chloride Reagents

Trimethylsilylacetylene (3 mmol) was deprotonated with n-butyl lithium (3 mmol) at −78° C. in THF and warmed slowly to room temperature. Excess ZnCl₂ (650 mg) in 5 mL of THF was transferred into the solution via canula. Pd(dppf) (3 mg) was prepared by stirring a suspension of Pd(dppf)Cl₂ in THF over Mg turnings for 20 min. and transferred into the solution by canula. The entire reaction mixture was transferred to a dry 100 mL Schlenk tube containing 340 mg of 5,15-dibromo-10,20-diphenylporphyrin. The solution was heated to 40° C. and left sealed overnight. TLC of the reaction mixture after 18 h shows a mixture of fluorescent products. The mixture was quenched with aqueous ammonium chloride, extracted with CH₂Cl₂, and dried over CaCl₂. The solvent was evaporated to dryness and chromatography was carried out with 1:1 CH₂Cl₂:hexane as eluant. The majority of the material was collected in two bands which proved to be [5-(2-trimethylsilylethynyl)-10,20-diphenylporphinato]zinc and [5,15-bis(2-trimethylsilylethynyl)-10,20-diphenylporphinato]-zinc. The two products were isolated in 83% overall yield.

B. Using Organotrialkyltin Reagents

5,15-Dibromo-10,20-diphenylporphyrin is placed in a dry 100 mL Schlenk tube and dissolved in 30 mL of THF. A solution of vinyltributyltin (3 mmol) in 5 mL THF is added to the reaction mixture. Pd(dppf) (3 mg) is prepared by stirring a suspension of Pd(dppf)Cl₂ in THF over Mg turnings for 20 min. and is transferred into the reaction mixture by canula. The solution is stirred overnight, quenched with aqueous ammonium chloride, extracted with CH₂Cl₂, and dried over CaCl₂. The solvent is evaporated to dryness and chromatography is carried out with 1:1 CH₂Cl₂:hexane as eluant. The product, 5,15-diphenyl-10,20-divinylprophyrin, elutes in one band and is isolated in 90% yield.

### EXAMPLE 14

### Coupling on Dilithialated Porphyrin Derivatives

A solution of N,N″-dilithio-5,15-dibromo-10,20-diphenylporphyrin (0.2 mmol) in 15 mL of THF is prepared generally according to the method of Arnold, J. Chem. Soc. Commun. 1990, 976. A solution of vinyltributyltin (2 mmol) in 5 mL THF is added to the reaction mixture. Pd(dppf) (3 mg) is prepared by stirring a suspension of Pd(dppf)Cl₂ in THF over Mg turnings for 20 min. and is transferred into the reaction mixture by canula. The solution is stirred overnight, and quenched with a solution of anhydrous NiCl₂ in THF. Aqueous ammonium chloride is added, the solution is extracted with CH₂Cl₂, and dried over CaCl₁. The solvent is evaporated to dryness and chromatography is carried out with 1:1 CH₂Cl₂:hexane as eluant. The product, [5,15-diphenyl-10,20-divinylporphinato]nickel, elutes in one band and is isolated in 90% yield.

### EXAMPLE 15

### Bis[(5,5′,-10,20-diphenylporphinato)zinc(II)]ethyne

Lithium bistrimethylsilylamide (1 mmol) was added to a solution of(5-ethynyl-10,20-diphenylporphinato)zinc(II) (50 mg, 0.1 mmol) in 20 ml THF to yield the (5-ethynyllithium-10,20-diphenylporphinato)zinc(II) reagent. (5-bromo-10,20-diphenylporphinato)zinc(II) (60 mg, 0.1 mmol) and 10 mg of Pd(PPh3)4 in 20 ml THF were added to this solution by canula. After completion of the metal-mediated cross-coupling reaction, chromatography was carried out on silica by using 9:1 hexane:THF as eluent. The first green band was isolated and evaporated to yield 77.2 mg of the product (yield=72%, based on (5-ethynyl-10,20-diphenylporphinato)zinc(II)). 1H NMR (250 MHz, CDCl3): □10.43 (d, 4 H, J=4.6 Hz), 10.03 (s, 2H), 9.21 (d, 4H, J=4.4Hz), 9.06 (d, 4H, J=4.5 Hz), 8.91 (d, 4H, J=4.4 Hz), 8.22 (m, 8H), 7.72 (m, 12H). Vis (CHCl3) 413.9 (4.96), 420.5 (4.97), 426.0 (4.96), 432.6 (4.92), 445.8 (4.89), 477.7 (5.1), 549.2 (4.15), 552.5 (4.14), 557.8 (4.15), 625.1 (4.09), 683.4 (4.37). FAB MS: 1070 (calcd 1070).

### EXAMPLE 16

### 5,15-Bis[[(5′-10,20-diphenylporphinato)zinc(II)]ethynyl]-[10,20-diphenylporphinato]zinc(II)

Pd(PPh3)4 (20 mg, 0.0173 mmol) and CuI (10 mg) were added to a solution of (5-bromo-10,20-diphenylporphinato)zinc(II) (120 mg, 0.2 mmol) in 20 ml THF. (5,15-diethynyl-10,20-diphenylporphinato)zinc(II) (57 mg, 0.1 mmol) and 0.35 ml of diethylamine in 20 ml THF were added to this solution by canula. After completion of the metal-mediated cross-coupling reaction, the precipitated product was isolated via filtration and then recrystalized from a pyridine-hexane mixture to give 66.5 mg of the product (yield=41% based on the (5,15-diethynyl-10,20-diphenylporphinato)zinc(II) starting material). 1H NMR (500 MHz, CDCl3): □10.86 (d, 4H, J=4.5 Hz), 10.78 (d, 4H, J=4.4 Hz), 10.39 (s, 2H), 9.50 (d, 4H, J=4.3Hz), 9.42 (d, 4H, J=4.4Hz), 9.32 (d, 4H, J=4.8Hz), 9.1 (d, 4H, J=4.0 Hz),8.52 (m, 4H), 8.47 (m, 8H), 7.89 (m, 6H), 7.85 (m, 12H). Vis (10:1 CHCl3:pyridine): 420.5 (4.84), 437.0 (4.72), 457.2 (4.66), 464.5 (4.66), 490.9 (4.85), 500.8 (4.95), 552.0 (3.99), 802.2(4.63). FAB MS: 1616 (calcd 1616).

### EXAMPLE 17

The following is a general procedure for the preparation of a conjugated compound composed of at least two covalently bound moieties in which the composite conjugated compound emits in the 650-2000 nm wavelength domain and possesses an emission dipole strength that is large with respect to the either of the covalently bound moieties (or alternatively, the sum of the emission dipole strength of each of the two covalently bound moieties).

A known fluorophore, lumophore, or phosphore which is known to emit light at a wavelength greater than or equal to 450 nm when optically or electrically pumped, is halogenated on its conjugated framework at a position that defines, or is spatially close to, either the head or tail of the lowest energy transition dipole. Those skilled in the art will recognize that experimental techniques such as pump-probe transient anisotropy measurements, can be utilized to determine the orientation of the lowest energy transition dipole on the molecular reference frame.

This halogenated fluorophore, lumophore, or phosphore is now subjected to a metal catalyzed cross-coupling reaction which results in the formation of an ethyne bond at the atomic position that bore the above said halogen moiety.

This ethynylated fluorophore, lumophore, or phosphore is now subjected to a second metal-catalyzed cross-coupling reaction with the above said halogenated fluorophore, lumophore, or phosphore under conditions appropriate to produce an ethyne-bridged bis(fluorophore, lumophore, or phosphore) compound, in which the ethyne moiety connects the two component emissive moieties along a vector that is defined by, or approximates, the head-to-tail alignment of their two respective transition dipoles.

Those skilled in the art will recognize that a known fluorophore, lumophore, or phosphore which is known to emit light at a wavelength greater than or equal to 450 nm when optically or electrically pumped, can be dihalogenated on its conjugated framework at the positions that define the head and tail of the lowest energy transition dipole. This species can be subjected to a metal-catalyzed cross-coupling reaction which results in the formation of ethyne bonds at the two atomic position that bore the above said halogen moieties.

Those skilled in the art will recognize that a combination of halogenated, dihalogenated, ethynylated, and diethynylated fluorophores, phosphores, or lumophores will enable the straightforward synthesis of dimeric, trimeric, tetrameric, and oligomeric species in which ethyne or butadiyne groups link the respective emissive units in a manner which provides head-to-tail alignment, or approximate head-to-tail alignment, of the low energy transition dipoles of the individual covalently bound moieties that comprise the conjugated compound.

### EXAMPLE 18

In the following examples, all manipulations were carried out under nitrogen previously passed through an O₂ scrubbing tower (Schweitzerhall R3-11 catalyst) and a drying tower (Linde 3-Å molecular sieves) unless otherwise stated. Air sensitive solids were handled in a Braun 150-M glove box. Standard Schlenk techniques were employed to manipulate air-sensitive solutions. CH₂Cl₂ and tetrahydrofuran (THF) were distilled from CaH₂ and K/4-benzoylbiphenyl, respectively, under N₂. N,N-Dimethylformamide (DMF) and benzonitrile were dried respectively over MgSO₄ and P₂O₅, and distilled under reduced pressure. All NMR solvents were used as received. ZnCl₂ was dried by heating under vacuum and stored under N₂. The catalysts Pd(PPh₃)₄ and tris(dibenzylideneacetone)dipalladium(0) (Pd₂dba₃), as well as triphenylarsine (AsPh₃) were purchased from Strem Chemicals and used as received. Meso- heptafluoropropyldipyrrylmethane ((a) Wijesekera, T. P. Can. J. Chem. 1996, 74, 1868-1871 (b) Nishino, N.; Wagner, R. W.; Lindsey, J. S. J. Org. Chem. 1996, 61, 7534-7544) and trimethylsilylpropynal (Kruithof, K. J. H.; Schmitz, R. F.; Klumpp, G. W. Tetrahedron 1983, 39, 3073-3081) were prepared according to the published procedures. The supporting electrolyte used in the electrochemical experiments, tetra-n-butylammonium hexafluorophosphate, was recrystallized two times from ethanol and dried under vacuum at 70° C. overnight prior to use.

Chemical shifts for ¹H NMR spectra are relative to tetramethylsilane (TMS) signal in the deuterated solvent (TMS, δ=0.00 ppm), while those for ¹⁹F NMR spectra are referenced to fluorotrichloromethane (CFCl₃, δ=0.00 ppm). All J values are reported in Hertz. Flash and size exclusion column chromatography were performed on the bench top, using respectively silica gel (EM Science, 230-400 mesh) and Bio-Rad Bio-Beads SX-1 as media. Mass spectra were acquired at the Mass Spectrometry Center at the University of Pennsylvania. MALDI-TOF mass spectroscopic data were obtained with a Perspective Voyager DE instrument in the Laboratory of Dr. Virgil Percec (Department of Chemistry, University of Pennsylvania). Samples were prepared as micromolar solutions in THF, and dithranol (Aldrich) was utilized as the matrix.

Instrumentation. Electronic spectra were recorded on an OLIS UV/vis/near-IR spectrophotometry system that is based on the optics of a Cary 14 spectrophotometer. Emission spectra were recorded on a SPEX Fluorolog luminescence spectrometer that utilized a T-channel configuration and PMT/InGaAs/Extended-InGaAs detectors; these spectra were corrected using a calibrated light source supplied by the National Bureau of Standards. NMR spectra were recorded on either 200 MHz AM-200, 250 MHz AC-250, or 500 MHz AMX-500 Brüker spectrometers. Cyclic voltammetric measurements were carried out on an EG&G Princeton Applied Research model 273A Potentiostat/Galvanostat. The electrochemical cell used for these experiments utilized a platinum disk working electrode, a platinum wire counter electrode, and a saturated calomel reference electrode (SCE). The reference electrode was separated from the bulk solution by a junction bridge filled with the corresponding solvent/supporting electrolyte solution. The ferrocene/ferrocenium redox couple was utilized as an internal potentiometric standard.

All electronic structure calculations were carried out using the GAUSSIAN 98 programs (Frisch, et al.,. Gaussian 98, Revision A.9; Gaussian, Inc: Pittsburgh, Pa., 1998. Geometry optimizations were performed using the semiempirical PM3 method. In order to minimize computational effort, the solubilizing phenyl substituents of the tris[(porphinato)zinc(II)] structures were replaced by hydrogen atoms, while C₃F₇ groups were replaced by C₂F₅. The models for the conjugated DDD and DAD tris[(porphinato)zinc(II)] compounds were optimized respectively within D₂ₕ and C₂ₕ symmetry constraints.

9-Methoxy-1,4,7-trioxanonyltosylate (1). p-Toluenesulfonyl chloride (17.69 g, 9.28×10⁻² mol) was dissolved in 50 ml of dry pyridine and cooled to −5 □C. Triethylene glycol monomethyl ether (13.50 ml, 8.44×10⁻² mol) was added dropwise to the solution, and the reaction mixture was stirred under N₂ for 4 h at −5 □C. The reaction mixture was poured onto ice and extracted three times with CH₂Cl₂. The combined organic layers were washed with 6M HCl, saturated aq. NaCl, and dried over Na₂SO₄. The solvent was evaporated to give a viscous oil. Yield=26.09 g (97%, based on 13.50 ml of triethylene glycol monomethyl ether). ¹H NMR (250 MHz, CDCl₃): δ7.80 (d, 2H, J=8.2 Hz, Ph—H), 7.35 (d, 2H, J=8.2 Hz, Ph—H), 4.16 (t, 2H, J=4.8 Hz, —O—CH₂—C), 3.69 (t, 2H, J=4.8 Hz, —O—CH₂—C), 3.61 (m, 6H, —O—CH₂—C), 3.53 (m, 2H, —O—CH₂—C), 3.38 (s, 3H, —OCH₃), 2.45 (s, 3H, —CH₃). CI MS m/z: 319 [(M+H)⁺] (calcd 319).

3,5-Bis(9-methoxy-1,4,7-trioxanonyl)benzaldehyde (2). 3,5-Dihydroxybenzaldehyde (3.052 g, 2.21×10⁻² mol), K₂CO₃ (9.00 g, 6.51×10⁻² mol) and 60 ml of dry DMF were added to a two-neck 200 ml flask, and the mixture stirred under N₂. A solution of 1 (16.53 g, 5.19×10⁻² mol) in 40 ml of dry DMF was added to the reaction mixture, following which it was refluxed for 1 h, cooled, diluted with 100 ml H₂O, and extracted several times with CH₂Cl₂. The combined organic layers were washed with water, saturated aq. NaCl, and dried over Na₂SO₄. After the solvent was evaporated, the residue was chromatographed on silica gel using 50:1 CH₂Cl₂:MeOH as the eluent. Yield=6.779 g (71%, based on 3.052 g of 3,5-dihydroxybenzaldehyde). ¹H NMR (250 MHz, CDCl₃): δ9.88 (s, 1H, —CHO), 7.02 (d, 2H, J=2.3 Hz, o-Ph—H), 6.76 (t, 1H, J=2.3 Hz, p-Ph—H), 4.16 (m, 4H, —O—CH₂—C), 3.87 (m, 4H, —O—CH₂—C), 3.75 (m, 4H, —O—CH₂—C), 3.67 (m, 8H, —O—CH₂—C), 3.56 (m, 4H, —O—CH₂—C), 3.38 (s, 6H, —OCH₃). CI MS m/z: 431 [(M+H)⁺] (calcd 431).

5,15-Bis[3,5-bis(9-methoxy-1,4,7-trioxanonyl)phenyl]porphyrin (3). 2,2′-Dipyrrylmethane (2.29 g, 1.57×10⁻² mol) and 2 (6.70 g, 1.56×10⁻² mol) were dissolved 2.7 L of dry CH₂Cl₂. The solution was purged with N₂ for 20 min before trifluoroacetic acid (0.30 ml, 3.89×10⁻³ mol) was added via syringe. The reaction mixture was stirred for 17 h at room temperature in the dark under N₂. DDQ (5.35 g, 2.36×10⁻² mol) was then added to the reaction mixture, and the solution stirred for an additional 2 h. The solvent was evaporated, and the residue chromatographed on silica gel using 30:1 CH₂Cl₂:MeOH as the eluent. Yield=2.820 g (33%, based on 6.70 g of 2). ¹H NMR (250 MHz, CDCl₃): δ10.31 (s, 2H, meso-H), 9.38 (d, 4H, J=4.6 Hz, δ-H), 9.15 (d, 4H, J=4.7 Hz, δ-H), 7.46 (d, 4H, J=2.2 Hz, o-Ph—H), 6.97 (t, 2H, J=2.2 Hz, p-Ph—H), 4.34 (m, 8H, —O—CH₂—C), 3.96 (m, 8H, —O—CH₂—C), 3.79 (m, 8H, —O—CH₂—C), 3.71 (m, 8H, —O—CH₂—C), 3.64 (m, 8H, —O—CH₂—C), 3.50 (m, 8H, —O—CH₂—C), 3.32 (s, 12H, —OCH₃), −2.03 (s, 2H, N—H). Vis (CH₂Cl₂): λₘₐₓ 407, 504, 537, 573, 629 nm. ESI MS m/z: 1133.5267 [(M+Na)⁺] (calcd for C₆₀H₇₈N₄O₁₆ 1133.5310).

5,15-Dibromo-10,20-bis[3,5-bis(9-methoxy-1,4,7-trioxanonyl)phenyl]porphyrin (4). Compound 3 (2.655 g, 2.39×10⁻³ mol) was dissolved in 300 ml of chloroform and cooled to −5 □C. Pyridine (2.0 ml) and N-bromosuccinimide (0.893 g, 5.02×10⁻³ mol) were added directly to the reaction mixture, and the reaction was followed by TLC (30:1 CHCl₃:MeOH). After 20 min, the reaction mixture was poured into water; the organic layer was separated, dried over Na₂SO₄, and evaporated. The residue was chromatographed on silica gel using 30:1 CHCl₃:MeOH as the eluant. Yield=2.950 g (97%, based on 2.655 g of the porphyrin starting material). ¹H NMR (250 MHz, CDCl₃): δ9.60 (d, 4H, J=4.9 Hz, β-H), 8.92 (d, 4H, J=4.9 Hz, β-H), 7.35 (d, 4H, J=2.3 Hz, o-Ph—H), 6.95 (t, 2H, J=2.2 Hz, p-Ph—H), 4.31 (m, 8H, —O—CH₂—C), 3.95 (m, 8H, —O—CH₂—C), 3.78 (m, 8H, —O—CH₂—C), 3.70 (m, 8H, —O—CH₂—C), 3.63 (m, 8H, —O—CH₂—C), 3.50 (m, 8H, —O—CH₂—C), 3.32 (s, 12H, —OCH₃), −2.44 (s, 2H, N—H). Vis (CH₂Cl2): λₘₐₓ 424, 520, 556, 599, 658 nm. ESI MS m/z : 1289.3452 [(M+Na)⁺] (calcd for 1289.3520).

(5,15-Dibromo-10,20bis[3,5-bis(9-methoxy-1,4,7-trioxanonyl)phenyl]porphinato)zinc(II) (5). Compound 4 (2.856 g, 2.25×10⁻³ mol) was dissolved in 200 ml of chloroform and refluxed. Zinc acetate dihydrate (1.23 g, 5.60×10⁻³ mol) in 50 ml of methanol was gradually added, and the reaction mixture was refluxed for an additional 2 h. After cooling to an ambient temperature, the solvent was evaporated, and the residue chromatographed on silica gel using 30:1 CHCl₃:MeOH as the eluent. Yield=2.914 g (97%, based on 2.856 g of the porphyrin starting material). ¹H NMR (250 MHz, CDCl₃): δ9.68 (d, 4H, J=4.7 Hz, β-H), 8.98 (d, 4H, J=4.7 Hz, β-H), 7.45 (d, 4H, J=2.3 Hz, o-Ph—H), 6.92 (t, 2H, J=2.2 Hz, p-Ph—H), 4.32 (m, 8H, —O—CH₂—C), 3.88 (m, 8H, —O—CH₂—C), 3.65 (m, 8H, —O—CH₂—C), 3.48 (m, 8H, —O—CH₂—C), 3.03 (m, 8H, —O—CH₂—C), 2.76 (m, 8H, —O—CH₂—C), 2.58 (s, 12H, —OCH₃). Vis (CH₂Cl₂): λₘₐₓ 426,559, 601 nm. ESI MS m/z: 1351.2683 [(M+Na)⁺] (calcd for 1351.2656).

(5,15-Bis[trimethylsilylethynyl]-10,20-bis[3,5-bis(9-methoxy-1,4,7-trioxanonyl)phenyl]porphinato)zinc(II) (6). Dry THF (20 mL) and (trimethylsilyl)acetylene (0.36 ml, 2.5×10⁻³ mol) were added to a 100 mL Schlenk tube, cooled to −78 ␣C, and stirred. Methyl lithium (1.4 M solution in diethyl ether, 1.80 ml, 2.52×10⁻³ mol) was added to the solution; after stirring for 30 min, the solution was warmed to room temperature, and ZnCl₂ (0.734 g, 5.39×10⁻³ mol) in 30 ml of dry THF was transferred into the reaction mixture and stirred for 10 min. This solution was canula transferred under N₂ to a Schlenk tube containing 5 (0.224 g, 1.68×10⁻⁴ mol) and Pd(PPh₃)₄ (0.031 g, 2.7×10⁻⁵ mol) in 30 ml of dry THF, and stirred at 60 □C for 16 h. The reaction mixture was then quenched with water and extracted with CHCl₃, following which the organic layer was washed with water, dried over CaCl₂, and evaporated. The crude product was chromatographed on silica gel using 30:1 CH₂Cl₂:MeOH as the eluent. Yield=0.206 g (90%, based on 0.224 g of the porphyrin starting material). ¹H NMR (250 MHz, CDCl₃): δ9.62 (d, 4H, J=4.6 Hz, β-H), 8.93 (d, 4H, J=4.7 Hz, β-H), 7.41 (d, 4H, J=2.2 Hz, o-Ph—H), 6.89 (t, 2H, J=2.2 Hz, p-Ph—H), 4.29 (m, 8H, —O—CH₂—C), 3.86 (m, 8H, —O—CH₂—C), 3.65 (m, 8H, —O—CH₂—C), 3.49 (m, 8H, —O—CH₂—C), 3.12 (m, 8H, —O—CH₂—C), 2.89 (m, 8H, —O—CH₂—C), 2.71 (s, 12H, —OCH₃), 0.61 (s, 18H, —Si—CH₃). Vis (CH₂Cl₂): λₘₐₓ (log ε) 437 (5.56), 578 (4.05), 629 (4.39) nm. ESI MS m/z: 1387.5280 [(M+Na)⁺] (calcd for 1387.5236).

(5,15-Diethynyl-10,20-bis[3,5-bis(9-methoxy-1,4,7-trioxanonyl)phenyl]porphinato) zinc (II) (7). Tetrabutylammonium fluoride (1 M in THF, 0.34 ml, 3.4×10⁻⁴mol) was added to a solution of 6 (0.156 g, 1.14×10⁻⁴ mol) in 30 ml of CH₂Cl₂ under N₂. The solution was extracted with water, dried over CaCl₂, and evaporated. The residue was chromatographed on silica gel using 25:1 CHCl₃:MeOH as the eluent. Yield=0.108 g (77%, based on 0.156 g of the porphyrin starting material). ¹H NMR (250 MHz, CDCl₃): δ9.67 (d, 4H, J=4.5 Hz, β-H), 8.97 (d, 4H, J=4.5 Hz, β-H), 7.44 (d, 4H, J=2.2 Hz, o-Ph—H), 6.90 (t, 2H, J=2.1 Hz, p-Ph—H), 4.29 (m, 8H, —O—CH₂—C), 3.86 (m, 8H, —O—CH₂—C), 3.65 (m, 8H, —O—CH₂—C), 3.49 (m, 8H, —O—CH₂—C), 3.12 (m, 8H, —O—CH₂—C), 2.89 (m, 8H, —O—CH₂—C), 2.71 (s, 12H, —OCH₃), 0.61 (s, 18H, —Si—CH₃). Vis (CH₂Cl₂): λₘₐₓ 429, 558, 609 nm. ESI MS m/z 1243.4413 [(M+Na)⁺] (calcd for 1243.4445).

3,3-Dimethyl-1-butyltosylate (8). p-Toluenesulfonyl chloride (17.35 g, 9.10×10⁻² mol) was dissolved in 50 ml of dry pyridine and cooled to 0 □C. 3,3-Dimethyl-1-butanol (11.0 ml, 9.10×10⁻² mol) was added dropwise, and the mixture was stirred under N₂ at 0 □C for 4 h, following which it was poured onto ice, and extracted three times with CH₂Cl₂. The combined organic layers were washed twice with 6 M HCl, saturated aq. NaHCO₃, saturated aq. NaCl, and dried over MgSO₄. The solvent was evaporated at room temperature to give a viscous oil. Yield=23.063 g (99%, based on 11.0 ml of 3,3-dimethyl-1-butanol). ¹H NMR (250 MHz, CDCl₃): δ7.80 (d, 2H, J=8.3 Hz, Ph—H), 7.35 (d, 2H, J=8.1 Hz, Ph—H), 4.09 (t, 2H, J=7.4 Hz, —O—CH₂—C), 2.46 (s, 3H, —CH₃), 1.58 (t, 2H, J=7.4 Hz, —OC—CH₂—C), 0.87 (s, 9H, —C—CH₃). CI MS m/z: 257 [(M+H)⁺] (calcd for 257).

3,5-Bis(3,3-dimethyl-1-butyloxy)benzaldehyde (9). 3,5-Dihydroxybenzaldehyde (4.008 g, 2.90×10⁻² mol), K₂CO₃ (8.016 g, 5.80×10⁻² mol) and 50 ml of dry DMF were stirred in a 100 mL round-bottom flask under N₂. Compound 8 (14.869 g, 5.80×10⁻² mol) was added, and the solution was heated at 80 □C for 13 h. The reaction mixture was then cooled, filtered, and evaporated, following which water was added to the residue, and the aqueous mixture extracted three times with CHCl₃. The combined organic layers were washed with 2% HCl solution, aq. NaHCO₃, aq. NaCl, and dried over MgSO₄. After removal of volatiles, the residue was chromatographed on silica gel with CHCl₃. Yield=7.314 g (82%, based on 4.008 g of 3,5-dihydroxybenzaldehyde). ¹H NMR (250 MHz, CDCl₃): δ9.85 (s, 1H, —CHO), 6.98 (d, 2H, J=2.3 Hz, o-Ph—H), 6.68 (t, 1H, J=2.3 Hz, p-Ph—H), 4.04 (t, 4H, J=7.3 Hz, —O—CH₂—C), 1.74 (t, 4H, J=7.3 Hz, —OC—CH₂—C), 1.00 (s, 18H, —C—CH₃). CI MS m/z: 307 [(M+H)⁺] (calcd 307).

5,15-Bis[3′,5′-di(3,3-dimethyl-1-butyloxy)phenyl]porphyrin (10). 2,2′-Dipyrrylmethane (1.604 g, 1.10×10⁻² mol) and 9 (3.342 g, 1.09×10⁻² mol) were dissolve 2.1 L of dry CH₂Cl₂. The solution was purged with N₂ for 20 min, following which trifluoroacetic acid (0.19 ml, 2.47×10⁻³ mol) was added via syringe. The reaction mixture was stirred in the dark for 22 h at room temperature under N₂. DDQ (3.70 g, 1.63×10⁻² mol) was then added, and the reaction mixture was stirred for an additional h. The solvent was evaporated, and the residue chromatographed on silica gel using CH₂Cl₂ as the eluent. Yield=2.234 g (47%, based on 3.342 g of 9). ¹H NMR (250 MHz, CDCl₃): δ10.31 (s, 2H, meso-H), 9.39 (d, 4H, J=4.7 Hz, β-H), 9.19 (d, 4H, J=4.7 Hz, β-H), 7.43 (d, 4H, J=2.3 Hz, o-Ph—H), 6.91 (t, 2H, J=2.2 Hz, p-Ph—H), 4.21 (t, 8H, J=7.4 Hz, —O—CH₂—C), 1.86 (t, 8H, J=7.4 Hz, —OC—CH₂—C), 1.00 (s, 36H, —C—CH₃), −2.06 (s, 2H, N—H). Vis (CH₂Cl₂): λₘₐₓ 408, 503, 535, 574, 628 nm. ESI MS m/z: 863.5494 [(M+H)⁺] (calcd 863.5476).

5-Bromo-10,20-bis[3′,5′-bis(3,3-dimethyl-1-butyloxy)phenyl]porphyrin (11). Compound 10 (1.667 g, 1.93×10⁻³ mol) was dissolved in 300 ml of CHCl₃ and cooled to −5 □C. Pyridine (2 mL) and N-bromosucciimide (0.345 g, 1.94×10⁻³ mol) were then added, and the reaction was followed by TLC. After 15 min, the reaction mixture was poured into water; the organic layer was separated, dried over Na₂SO₄, filtered, and evaporated. The residue was chromatographed on silica gel using 3:2 CHCl₃:hexanes as the eluent. Two products were recovered: 5,15-dibromo-10,20-bis[3′,5′-bis(3,3-dimethyl-1-butyloxy)phenyl]porphyrin (0.390 g, 20%) and the target compound (1.058 g, 58%, based on 1.667 g of the porphyrin starting material). ¹H NMR (250 MHz, CDCl₃): δ10.16 (s, 1H, meso-H), 9.73 (d, 2H, J=4.9 Hz, β-H), 9.28 (d, 2H, J=4.6 Hz, β-H), 9.08 (d, 2H, J=4.7 Hz, β-H), 9.07 (d, 2H, J=4.8 Hz, β-H), 7.37 (d, 4H, J=2.1 Hz, o-Ph—H), 6.90 (t, 2H, J=1.8 Hz, p-Ph—H), 4.20 (t, 8H, J=7.4 Hz, —O—CH₂—C), 1.85 (t, 8H, J=7.4 Hz, —OC—CH₂—C), 1.00 (s, 36H, —C—CH₃), −2.20 (s, 2H, N—H). Vis (CH₂Cl₂): λₘₐₓ 417,511,544, 587, 644 nm. ESI MS m/z: 941.4597 [(M+H)⁺] (calcd 941.4580).

(5-Bromo-10,20-bis[3′,5′-bis(3,3-dimethyl-1-butyloxy)phenyl]porphinato)zinc(II) (12). Compound 11 (1.315 g, 1.40×10⁻³ mol) was dissolved in 150 ml of CHCl₃ and refluxed. Zinc acetate dihydrate (0.770 g, 3.51×10⁻³ mol) in 25 ml of methanol was added gradually, and the reaction mixture was refluxed for 2 h. After cooling and removal of volatiles, the residue was chromatographed on silica gel using 15:1 hexanes:THF as the eluent. Yield=1.348 g (96%, based on 1.315 g of the porphyrin starting material). ¹H NMR (250 MHz, CDCl₃): δ10.23 (s, 1H, meso-H), 9.81 (d, 2H, J=4.8 Hz, β-H), 9.37 (d, 2H, J=4.6 Hz, β-H), 9.18 (d, 2H, J=4.6 Hz, β-H), 9.17 (d, 2H, J=4.7 Hz, β-H), 7.38 (d, 4H, J=2.2 Hz, o-Ph—H), 6.90 (t, 2H, J=2.2 Hz, p-Ph—H), 4.19 (t, 8H, J=7.4 Hz, —O—CH₂—C), 1.85 (t, 8H, J=7.4 Hz, —OC—CH₂—C), 1.00 (s, 36H, —C—CH₃). Vis (CH₂Cl₂): λₘₐₓ 418, 547, 580 nm. ESI MS m/z: 1002.3601 (M⁺) (calcd 1002.3638).

(5-Trimethylsilylethynyl-10,20-bis[3′,5′-bis(3,3-dimethyl-1-butyloxy)phenyl]porphinato)zinc(II) (13). THF (20 ml) and (trimethylsilyl)acetylene (0.56 ml, 3.96×10⁻³ mol) were added to a 100 mL Schlenk tube, stirred, and cooled to −78 □C. Methyl lithium (1.4 M solution in diethyl ether, 2.90 ml, 4.06×10⁻³ mol) was added, and the solution stirred for 30 min. After warming to room temperature, ZnCl₂ (1.095 g, 8.03×10⁻³ mol) in 50 ml of dry THF was transferred to the reaction mixture by canula. After stirring for 10 min, the reaction mixture was transferred to a 250 mL Schlenk tube containing 12 (0.404 g, 4.02×10⁻⁴mol) and Pd(PPh₃)₄ (0.069 g, 5.97×10⁻⁵ mol) and 40 ml of dry THF. The mixture was stirred under N₂ at 60 □C for 16 h, following which it was quenched with water, extracted with CH₂Cl₂, washed with water, dried over CaCl₂, and evaporated. The crude product was chromatographed on silica gel using 10:1 hexanes:THF as the eluent. Yield=0.404 g (98%, based on 0.404 g of the porphyrin starting material). ¹H NMR (250 MHz, CDCl₃): δ10.18 (s, 1H, meso-H), 9.78 (d, 2H, J=4.5 Hz, β-H), 9.33 (d, 2H, J=4.5 Hz, H), 9.14 (d, 2H, J=4.7 Hz, β-H), 9.13 (d, 2H, J=4.5 Hz, β-H), 7.37 (d, 4H, J=2.2 Hz, o-Ph—H), 6.88 (t, 2H, J=2.2 Hz, p-Ph—H), 4.19 (t, 8H, J=7.4 Hz, —O—CH₂—C), 1.84 (t, 8H, J=7.4 Hz, —OC—CH₂—C), 1.00 (s, 36H, —C—CH₃), 0.62 (s, 9H, —Si—CH₃). Vis (CH₂Cl₂): λₘₐₓ (log ε) 427 (5.51), 554 (4.15), 594 (3.70) nm. ESI MS m/z: 1021.5013 [(M+H)⁺] (calcd 1021.5005).

(5-Ethynyl-10,20-bis[3′,5′-bis(3,3-dimethyl-1-butyloxy)phenyl]porphinato)zinc(II) (14). Tetrabutylammonium fluoride (1 M in THF, 0.73 ml, 7.3×10⁻⁴mol) was added to a solution of 13 (0.375 g, 3.67×10⁻⁴mol) in 40 ml of CH₂Cl₂ under N₂. The reaction mixture was stirred for 10 min, quenched with water, extracted with CH₂Cl₂, and dried over CaCl₂. After the solvent was evaporated, the residue was chromatographed on silica gel using 10:1 hexanes:THF as the eluent. Yield 0.340 g (97%, based on 0.375 g of the porphyrin starting material). ¹H NMR (250 MHz, CDCl₃): δ10.22 (s, 1H, meso-H), 9.79 (d, 2H, J=4.7 Hz, β-H), 9.35 (d, 2H, J=4.5 Hz, β-H), 9.17 (d, 2H, J=4.7 Hz, β-H), 9.15 (d, 2H, J=4.6 Hz, β-H), 7.37 (d, 4H, J=2.2 Hz, o-Ph—H), 6.88 (t, 2H, J=2.6 Hz, p-Ph—H), 4.18 (t, 8H, J=7.4 Hz, —O—CH₂—C), 4.15 (s, 1H, —CC—H), 1.84 (t, 8H, J=7.4 Hz, —OC—CH₂—C), 1.00 (s, 36H, —C—CH₃). Vis (CH₂Cl₂): λₘₐₓ 422, 552, 590 nm. ESI MS m/z: 949.4595 [(M+H)⁺] (calcd 949.4611).

Bis[(5,5′-10,20-bis[3,5-bis(3,3-dimethyl-1-butyloxy)phenyl]porphinat)zinc(II)]ethyne (DD). Compounds 12 (0.0696 g, 6.92×10⁻⁵ mol) and 14 (0.066 g, 6.94×10⁻⁵ mol), 20 ml of dry THF, and 2.0 ml of triethylamine were added to a 50 mL Schlenk tube. Pd₂(dba)₃ (0.019 g, 2.07×10⁻⁵ mol) and AsPh₃ (0.051 g, 1.67×10⁻⁴ mol) were transferred to the Schlenk tube in a dry box, following which the solution was degassed by three successive freeze-pump-thaw cycles. The reaction mixture was stirred at 45 □C for 10.5 h, after which time the solvent was evaporated, and the residue chromatographed on silica gel using 10:1 hexanes:THF as the eluent. Yield=0.115 g (89%, based on 0.0696 g of 12). ¹H NMR (250 MHz, CDCl₃): δ10.48 (d, 4H, J=4.7 Hz, β-H), 10.19 (s, 2H, meso-H), 9.37 (d, 4H, J=4.4 Hz, β-H), 9.35 (d, 4H, J=4.5 Hz, β-H), 9.19 (d, 4H, J=4.5 Hz, β-H), 7.46 (d, 8H, J=2.2 Hz, o-Ph—H), 6.91 (t, 4H, J=2.2 Hz, p-Ph—H), 4.22 (t, 16H, J=7.4 Hz, —O—CH₂—C), 1.86 (t, 16H, J=7.4 Hz, —OC—CH₂—C), 1.01 (s, 72H, —C—CH₃). Vis (CH₂): λₘₐₓ (log ε) 399 (5.06), 404 (5.06), 426 (5.03), 439 (4.96), 476 (5.42), 539 (4.19), 558 (4.23), 669 (4.62) nm. MALDI-TOF MS m/z: 1871.65 (M⁺) (calcd 1870.8906).

[(5,-10,20-bis[3,5-bis(3,3-dimethyl-1-butyloxy)phenyl]porphinato)zinc(II)]-[(5′,-15′-bromo-10′,20′-bis[3,5-bis(3,3-dimethyl-1-butyloxy)phenyl]porphinato)zinc(II)]ethyne (DD-Br) and 5,15-bis[[5′,-10′,20′-bis[3,5-di(3,3-dimethyl-1-butyloxy)phenyl]porphinato)zinc(II)]ethynyl]-10,20-bis[3,5-di(9-methoxy-1,4,7-trioxanonyl)phenyl]porphinato)zinc(II) (DDD). Compound 5 (0.692 g, 5.19×10⁻⁴ mol), Pd(PPh₃)₄ (0.046 g, 3.98×10⁻⁵ mol), and CuI (0.023 g, 1.21×10⁻⁴ mol) were added to a 100 mL Schlenk tube. Following the addition of 40 ml of dry THF, a solution of 14 (0.307 g, 3.23×10⁻⁴mol) and diethylamine (0.60 ml, 5.80×10⁻³ mol) in 30 ml of dry THF was added via canula. The reaction mixture was stirred under N₂ at 55 □C for 65 h, after which time it was quenched with water. The organic layer was extracted with CHCl₃, washed with water, dried over CaCl₂, and evaporated. The residue was chromatographed on silica gel using 1:1 hexanes:THF as the eluent. Two products were recovered: DD-Br 0.318 g (45%, based on 0.307 g of 14) and DDD 0.193 g (24%). DD-Br: ¹H NMR (250 MHz, CDCl₃): δ10.49 (d, 2H, J=4.6 Hz, β-H), 10.39 (d, 2H, J=4.7 H, β-H), 10.20 (s, 1H, meso-H), 9.67 (d, 2H, J=4.8 Hz, β-H), 9.36 (d, 2H, J=4.4 Hz, β-H), 9.35 (d, 2H, J=4.4 Hz, β-H), 9.17 (d, 2H, J=4.5 Hz, β-H), 9.17 (d, 2H, J=4.6 Hz, β-H), 8.99 (d, 2H, J=4.7 Hz, β-H), 7.51 (d, 4H, J=2.1 Hz, o-Ph—H), 7.45 (d, 4H, J=2.2 Hz, o-Ph—H), 6.91 (t, 2H, J=2.0 Hz, p-Ph—H), 6.88 (t, 2H, J=2.7 Hz, p-Ph—H), 4.30 (m, 8H, —O—CH₂—C), 4.20 (t, 8H, J=7.4 Hz, —O—CH₂—C), 3.83 (m, 8H, —O—CH₂—C), 3.61 (m, 8H, —O—CH₂—C), 3.44 (m, 8H, —O—CH₂—C), 3.07 (m, 8H, —O—CH₂—C), 2.82 (m, 8H, —O—CH₂—C), 2.65 (s, 12H, —OCH₃), 1.84 (t, 8H, J=7.3 Hz, —OC—CH₂—C), 0.99 (s, 36H, —C—CH₃). Vis (CH₂Cl₂): λₘₐₓ 410, 429, 441, 479, 546, 686 nm. MALDI-TOF MS m/z: 2198.1 (M⁺) (calcd 2196.80). DDD: ¹H NMR (250 MHz, CDCl₃): δ10.53 (d, 4H, J=4.6 Hz, β-H), 10.41 (d, 4H, J=4.5 H, β-H), 10.18 (s, 2H, meso-H), 9.37 (d, 4H, J=4.7 Hz, β-H), 9.35 (d, 4H, J=5.6 Hz, β-H), 9.20 (d, 4H, J=4.6 Hz, β-H), 9.16 (d, 4H, J=4.5 Hz, β-H), 7.57 (d, 4H, J=2.0 Hz, o-Ph—H), 7.44 (d, 8H, J=2.1 Hz, o-Ph—H), 6.84 (t, 6H, J=2.1 Hz, p-Ph—H), 4.23 (m, 8H, —O—CH₂—C), 4.17 (t, 16H, J=7.4 Hz, —O—CH₂—C), 3.74 (m, 8H, —O—CH₂—C), 3.50 (m, 8H, —O—CH₂—C), 3.33 (m, 8H, —O—CH₂—C), 2.99 (m, 8H, —O—CH₂—C), 2.78 (m, 8H, —O—CH₂—C), 2.61 (s, 12H, —O—CH₃), 1.81 (t, 16H, J=7.4 Hz, —OC—CH₂—C), 0.97 (s, 72H, —C—CH₃). Vis (CH₂Cl₂): λₘₐₓ (log ε) 410 (5.33), 490 (5.54), 542 (4.34), 563 (4.39), 742 (4.99) nm. MALDI-TOF MS m/z: 3066.6 (M⁺) (calcd 3065.33).

5,15-Bis[[15″,-(5′,-10′,20′-bis[3,5-bis(3,3-dimethyl-1-butyloxy)phenyl]porphinato)zinc(II)]-[(5″,-10″,20″-bis[3,5-di(9-methoxy-1,4,7-trioxanonyl)phenyl]porphinato)zinc(II)]ethynelethynyl]-10,20-bis[3,5-di(9-methoxy-1,4,7-trioxanonyl)phenyl]porphinato)zinc(II) (DDDDD). DD-Br (0.259 g, 1.18×10⁻⁴mol), 7 (0.082 g, 6.71×10⁻⁵ mol), and diethylamine (0.20 ml, 1.93×10⁻³ mol) were added to a 100 ml Schlenk tube and dissolved in 40 ml of dry THF. Following the addition of Pd(PPh₃)₄ (0.012 g, 1.04×10⁻⁵ mol) and CuI (0.006 g, 3.2×10⁻⁵ mol) in a dry box, the solution was degassed by three freeze-pump-thaw cycles. The reaction solution was stirred under N₂ at 50 □C for 84 h. The reaction was then quenched with water; the organic layer was extracted with CHCl₃, washed with water, and dried over CaCl₂. Following evaporation of volatiles, the residue was chromatographed on silica gel using 30:1 CHCl₃:MeOH as the eluent. The product mixture was separated by preparative size exclusion chromatography (BioRad Bio-Beads SX-1 packed in THF, gravity flow), after which the product pentamer was re-chromatographed on silica gel using 20:1 CHCl₃:MeOH as the eluent. Yield=0.163 g (51%, based on 0.259 g of DD-Br). ¹H NMR (500 MHz, CDCl₃): δ10.51 (d, 4H, J=4.6 Hz, β-H), 10.47 (m, 8H, β-H), 10.40 (d, 4H, J=4.5 Hz, β-H), 10.15 (s, 2H, meso-H), 9.34 (d, 4H, J=4.6 Hz, β-H), 9.31 (d, 4H, J=4.6 Hz, β-H), 9.26 (d, 4H, J=4.5 Hz, β-H), 9.22 (d, 4H, J=4.1 Hz, β-H), 9.17 (d, 4H, J=4.5 Hz, β-H), 9.12 (d, 4H, J=4.5 Hz, β-H), 7.51 (br s, 8H, o-Ph—H), 7.47 (Br s, 4H, o-Ph—H), 7.40 (br s, 8H, o-Ph—H), 6.76 (br s, 4H, p-Ph—H), 6.66 (br s, 4H, p-Ph—H), 6.50 (br s, 2H, p-Ph—H), 4.12 (t, 16H, J=6.7 Hz, —O—CH₂—C), 4.04 (br s, 16H, —O—CH₂—C), 3.91 (Br s, 8H, —O—CH₂—C), 3.52 (br s, 16H, —O—CH₂—C), 3.41 (br s, 8H, —O—CH₂—C), 3.31 (br s, 16H, —O—CH₂—C), 3.22 (br s, 8H, —O—CH₂—C), 3.15 (br s, 16H, —O—CH₂—C), 3.08 (br s, 8H, —O—CH₂—C), 2.90 (br s, 16H, —O—CH₂—C), 2.86 (br s, 8H, —O—CH₂—C), 2.72 (br s, 24H, —O—CH₂—C), 2.58 (s, 36H, —OCH₃), 1.76 (t, 16H, J=7.2 Hz, OC—CH₂—C), 0.93 (s, 72H, —C—CH₃). Vis (CH₂Cl₂): λₘₐₓ (log ε) 412 (5.45), 490 (5.68), 809 (5.27) nm. MALDI-TOF MS m/z: 5454.6 (M⁺) (calcd 5454.21).

5,15-Bis(trimethylsilylethynyl)-10,20-bis(heptafluoropropyl)porphyrin (15). Meso-heptafluoropropyldipyrrylmethane (2.131 g, 6.78×10⁻³ mol) and trimethylsilylpropynal (0.856 g, 6.78×10⁻³ mol) were dissolved in 500 ml of dry CH₂Cl₂. The solution was degassed with N₂ for 20 min and cooled to −5 □C. BF₃.Et₂O (0.17 ml, 1.34×10⁻³ mol) was added via syringe and the reaction mixture stirred for 2 h. The solution was then warmed to room temperature and stirred for an additional 11 h, following which DDQ (2.30 g, 1.01×10⁻² mol) was added. After stirring for 1 h, the solvent was evaporated and the residue chromatographed on silica gel using 3:1 hexanes:CHCl₃ as the eluent. Yield=0.314 g (11%, based on 2.131 g of meso-heptafluoropropyldipyrrylmethane). ¹H NMR (250 MHz, CDCl₃): δ9.82 (d, 4H, J=5.1 Hz, β-H), 9.46 (br s, 4H, β-H), 0.65 (s, 18H, —Si—CH₃), −2.08 (s, 4H, N—H). ¹⁹F NMR (188 MHz, CDCl₃): δ−79.6 (t, 6F), −83.9 (m, 4F), −120.8 (s, 4F). Vis (CH₂Cl₂): λₘₐₓ 435, 533, 567, 618, 678 nm. ESI MS m/z: 839.1719 [(M+H)⁺] (calcd 839.1707).

[5,15-Bis(trimethylsilylethynyl)-10,20bis(heptafluoropropyl)porphinato]zinc(II) (16). Compound 15 (0.496 g, 5.91×10⁻⁴ mol) was dissolved in 100 ml of CHCl₃ and refluxed. Zinc acetate dihydrate (0.260 g, 1.18×10⁻³ mol) in 15 ml of methanol was gradually added; the solution was refluxed for 2.5 h, and subsequently cooled and evaporated. The residue was chromatographed on silica gel using 15:1 hexanes:THF as the eluent. Yield=0.506 g (95%, based on 0.496 g of the porphyrin starting material). ¹H NMR (250 MHz, CDCl₃): δ9.77 (d, 4H, J=5.0 Hz, β-H), 9.53 (br s, 4H, β-H), 0.65 (s, 18H, —Si—CH₃). ¹⁹F NMR (188 MHz, CDCl₃): δ−79.4 (s, 4F), −79.7 (m, 6F), −120.0 (s, 4F). Vis (THF): λₘₐₓ (log ε) 442 (5.71), 570 (4.05), 591 (4.15), 635 (3.39) nm. ESI MS m/z: 900.0769 (M⁺) (calcd 900.0764).

[5,15-Diethynyl-10,20-bis(heptafluoropropyl)porphinato]zinc(II) (17). Tetrabutylammonium fluoride (1 M in THF, 1.12 ml, 1.12×10⁻³ mol) was added to a solution of 16 (0.455 g, 5.04×10⁻⁴mol) in 50 ml of dry THF under N₂. The reaction mixture was stirred for 10 min, quenched with water, and extracted with CHCl₃, following which it was washed with water, dried over CaCl₂, and evaporated. The residue was chromatographed on silica gel using 15:1 hexanes:THF as the eluent. Yield=0.377 g (99%, based on 0.455 g of the porphyrin starting material). ¹H NMR (250 MHz, 1 drop pyridine-d₅ in CDCl₃): δ9.81 (d, 4H, J=4.9 Hz, β-H), 9.58 (br s, 4H, β-H), 4.20 (s, 2H, —CC—H). ¹⁹F NMR (188 MHz, 1 drop pyridine-d₅ in CDCl₃): δ−78.8 (s, 4F), −79.7 (s, 6F), −119.8 (s, 4F). Vis (THF): λₘₐₓ 435, 564, 582 nm. ESI MS m/z: 790.9659 [(M+Cl)⁺] (calcd 790.9662).

[(5,-10,20-bis[3,5-bis(3,3-dimethyl-1-butyloxy)phenyl]porphinato)zinc(II)]-[(5′,-15′-ethynyl-10′,20′-bis[10,20-bis(heptafluoropropyl)porphinato)zinc(II)]ethyne (DA-ethyne) and 5,15-bis[[5′,10′,20′-bis[3,5-di(3,3-dimethyl-1-butyloxy)phenyl]porphinato)zinc(II)]ethynyll-10,20-bis(heptafluoropropyl)porphinato]zinc(II) (DAD). Compounds 12 (0.120 g, 1.19×10⁻⁴ mol) and 17 (0.108 g, 1.43×10⁴ mol), THF (30 ml) and triethylamine (3.0 mL) were added to a 100 mL Schlenk tube. Following the addition of Pd₂(dba)₃ (16.3 mg, 1.78×10⁻⁵ mol) and AsPh₃ (43.7 mg, 1.43×10⁻⁴ mol) in a dry box, the solution was degassed via three freeze-pump-thaw cycles. The reaction mixture was stirred under N₂ at 35 □C for 7 h, and evaporated. The residue was chromatographed on silica gel using 10:1 hexanes:THF as the eluent. The recovered bis- and tris[porphinato]zinc(II) products were further purified by preparative size exclusion chromatography (BioRad Bio-Beads SX-1 packed in THF, gravity flow), followed by an additional round of silica gel chromatography that utilized 8:1 hexanes:THF as the eluent. Two products were recovered: DA-ethyne 0.100 g (50%, based on 0.120 g of 12) and DAD 0.031g (20%). DA-ethyne: ¹H NMR (250 MHz, 1 drop pyridine-d₅ in CDCl₃): δ10.56 (d, 2H, J=4.9 Hz, β-H), 10.42 (d, 2H, J=4.6 Hz, β-H), 10.15 (s, 1H, meso-H), 9.82 (d, 2H, J=4.9 Hz, β-H), 9.73 (br s, 2H, β-H), 9.60 (br s, 2H, β-H), 9.35 (d, 2H, J=4.6 Hz, β-H), 9.31 (d, 2H, J=4.5 Hz, β-H), 9.13 (d, 2H, J=4.5 Hz, β-H), 7.48 (d, 4H, J=2.2 Hz, o-Ph—H), 6.94 (t, 2H, J=2.2 Hz, p-Ph—H), 4.26 (t, 8H, J=7.4 Hz, —O—CH₂—C), 4.22 (s, 1H, —CC—H), 1.89 (t, 8H, J=7.4 Hz, —OC—CH₂—C), 1.03 (s, 36H, —C—CH₃). ¹⁹F NMR (188 MHz 1 drop pyridine-d₅ in CDCl₃): δ−78.9 (s, 4F), −79.6 (t, 6F), −119.8 (s, 4F). Vis (THF): λₘₐₓ (log ε) 429 (5.06), 450 (4.92), 471 (4.96), 484 (4.95), 550 (4.25), 593 (4.14), 688 (4.55) nm. MALDI-TOF MS nmz: 1678.64 (M⁺) (calcd 1678.44). DAD: ¹H NMR (250 MHz, 1 drop pyridine-d₅ in CDCl₃): δ10.56 (d, 4H, J=5.0 Hz, β-H), 10.45 (d, 4H, J=4.7 Hz, β-H), 10.15 (s, 2H, meso-H), 9.76 (br s, 4H, β-H), 9.36 (d, 4H, J=4.5 Hz, β-H), 9.32 (d, 4H, J=4.4 Hz, β-H), 9.14 (d, 4H, J=4.4 Hz, β-H), 7.49 (d, 8H, J=2.2 Hz, o-Ph—H), 6.94 (t, 4H, J=2.1 Hz, p-Ph—H), 4.27 (t, 16H, J=7.4 Hz, —O—CH₂—C), 1.90 (t, 16H, J=7.4 Hz, —OC—CH₂—C), 1.04 (s, 72H, —C—CH₃). ¹⁹F NMR (188 MHz, 1 drop pyridine-d₅ in CDCl₃): δ−78.9 (s, 4F), −79.6 (s, 6F), −119.7 (s, 4F). Vis (THF): λₘₐₓ (log ε) 432 (5.29), 506 (5.18), 566 (4.49), 593 (4.42), 735 (4.93) nm. MALDI-TOF MS: m/z 2602 (calcd 2600.87).

5,15-Bis[[15″,-(5′,-10′,20′-bis[3,5-bis(3,3-dimethyl-1-butyloxy)phenyl]porphinato)zinc(II)]-[(5″,-(10″,20″-bis(heptafluoropropyl)porphinato)zinc(II)]ethyne]ethynyl]-10,20-bis[3,5-di(9-methoxy-1,4,7-trioxanonyl)phenyl]porphinato)zinc(II) (DADAD). DA-ethyne (0.085 g, 5.05×10⁻⁵ mol), 5 (0.0344 g, 2.58×10⁻⁵ mol), dry THF (20 ml), and triethylamine (2.0 mL) were added to a 50 ml Schlenk tube. Following the addition of Pd₂(dba)₃ (3.6 mg, 3.93×10⁻⁶ mol) and AsPh₃ (9.7 mg, 3.17×10⁻⁵ mol) in a dry box, the solution was degassed via three freeze-pump-thaw cycles. The reaction mixture was stirred under N₂ at 40 □C for 18 h, and evaporated. The residue was chromatographed on silica gel using 1:1 hexanes:THF as the eluent. The recovered high molecular weight porphyrinic products were separated using preparative size exclusion chromatography (BioRad Bio-Beads SX-1 packed in THF, gravity flow); the isolated product band was subjected to an additional round of silica gel chromatography that utilized 25:1 CH₂Cl₂:MeOH as the eluent. Yield=0.027 g (24%, based on 0.085 g of DA-ethyne). ¹H NMR (250 MHz, 1 drop pyridine-d₅ in CDCl₃): δ10.58 (d, 8H, J=4.8 Hz, β-H), 10.46 (d, 4H, J=4.3 Hz, β-H), 10.40 (d, 4H, J=4.8 Hz, β-H), 10.16 (s, 2H, meso-H), 9.80 (br s, 8H, β-H), 9.37 (d, 4H, J=4.3 Hz, β-H), 9.33 (d, 4H, J=4.4 Hz, β-H), 9.29 (d, 4H, J=4.4 Hz, β-H), 9.15 (d, 4H, J=4.6 Hz, β-H), 7.62 (d, 4H, J=2.2 Hz, o-Ph—H), 7.50 (d, 8H, J=2.2 Hz, o-Ph—H), 7.06 (t, 2H, J=2.2 Hz, p-Ph—H), 6.95 (t, 4H, J=2.2 Hz, p-Ph—H), 4.46 (m, 8H, —O—CH₂—C), 4.28 (t, 16H, J=7.3 Hz, —O—CH₂—C), 4.05 (m, 8H, —O—CH₂—C), 3.87 (m, 8H, —O—CH₂—C), 3.77 (m, 8H, —O—CH₂—C), 3.70 (m, 8H, —O—CH₂—C), 3.55 (m, 8H, —O—CH₂—C), 3.34 (s, 12H, —OCH₃), 1.91 (t, 16H, J=7.3 Hz, —OC—CH₂—C), 1.05 (s, 72H, —C—CH₃). ¹⁹F NMR (188 MHz, 1 drop pyridine-d₅ in CDCl₃): □−79.0 (s, 8F), −79.6 (t, 12F), −119.7 (s, 8F). Vis (THF): λₘₐₓ (log ε) 430 (5.35), 507 (5.41), 595 (4.54), 798 (5.25) nm. MALDI-TOF MS m/z: 4525.51 (M⁺) (calcd 4525.29).

Those skilled in the art will appreciate that numerous changes and modifications may be made to the preferred embodiments of the invention and that such changes and modifications may be made without departing from the spirit of the invention. For example, it is believed that the methods of the present invention can be practiced using porphyrin-related compounds such as chlorins, phorbins, bacteriochlorins, porphyrinogens, sapphyrins, texaphrins, and pthalocyanines in place of porphyrins. It is also believed that, in addition to ethyne and butadiyne moities, the invention can be practiced using other moieties, including ethene, polyines, phenylene, thiophene, anene, or allene.

It is therefore intended that the appended claims cover all such equivalent variations as fall within the true spirit and scope of the invention.

What is claimed is:

1. A method comprising the steps of:
providing a conjugated compound comprising at least two covalently bound moieties; and
exposing said compound to an energy source for a time and under conditions effective to cause said compound to emit light that has a wavelength of 650-2000 nm and is of an intensity that is greater than a sum of light emitted by said moieties.

2. The method of claim 1 wherein said compound exhibits an integrated emission oscillator strength that is greater than a sum of emission oscillator strengths exhibited by said moieties.

3. The method of claim 1 wherein said moieties each include a conjugated ring system.

4. The method of claim 1 wherein at least one of said moieties is a laser dye, fluorophore, lumophore, or phosphore.

5. The method of claim 1 wherein at least one of said moieties is a porphyrin, porphycene, rubyrin, rosarin, hexaphyrin, supphyrin, chlorophyl, chlorin, phthalocynine, porphyrazine, bacteriochlorophyl, pheophytin, texaphyrin group or and their corresponding metalated derivatives.

6. The method of claim 1 wherein said moieties are bound by at least one carbon-carbon double bond, carbon-carbon triple bond, or a combination thereof.

7. The method of claim 6 wherein said bond is ethynyl, ethenyl, allenyl, butadiynyl, polyvinyl, thiophenyl, furanyl, pyrrolyl, p-dethylylarenyl or any conjugated hetrocycle that bears diethynyl, di(polyynynyl), divinyl, di(polyvinvyl), or di(thiophenyl) substituents.

8. The method of claim 1 wherein said moieties are bound by at least one imine, phenylene, thiophene, or amide, ether, thioether, ester, ketone, sulfone, or carbodiimide group.

9. A laser comprising:
a dye solution disposed in a resonant cavity, said solution comprising a compound of claim 1 and a non-aqueous solvent that is substantially unable to chemically react with said compound and to absorb and emit light at a wavelength at which said compound absorbs and emits light, and
a pumping energy source that produces stimulated emission in the dye solution.

10. A laser comprising
a solid body that includes a compound of claim 1 and a host polymer, the host polymer being unable to chemically react with said compound and unable to absorb and emit light at a wavelength at which said compound absorbs and emits light; and
an energy source that is coupled with said solid body and generates light in said solid body.

11. A laser comprising
a solid body that includes a compound of claim 1 and a host polymer, the host polymer being unable to chemically react with said compound and unable to absorb and emit light at a wavelength at which said compound absorbs and emits light; and
an energy source that is coupled with said host polymer and generates light in said host polymer.

12. An optical amplifier comprising a polymeric optical waveguide and a compound of claim 1.

13. A polymer grid comprising a body of electrically conducting organic polymer, said body having an open and porous network morphology and defining an expanded surface, area void-defining porous network, and an active electronic material located within at least a portion of the void spaces defined by the porous network, said active electronic material comprising a compound of claim 1.

14. The polymer grid of claim 13 wherein the conducting organic polymer comprises the compound of claim 1.

15. A polymer grid electrode comprising a body of electrically conducting organic polymer, electrically joined to an electrical connector, said body having an open and porous network morphology and defining an expanded surface area, void-defining porous network, and an active electronic material located within at least a portion of the void spaces defined by the porous network, said active electronic material comprising the compound of claim 1.

16. A solid state polymer grid triode comprising a first electrode and a second electrode spaced apart from one another with a polymer grid comprising a body of electrically conducting organic polymer said body having an open and porous network morphology and defining an expanded surface area void-defining porous network interposed between the first electrode and the second electrode
wherein the conducting organic polymer comprises the compound of claim 1.

17. A light-emitting polymer grid triode comprising a first electrode and a second electrode spaced apart from one another with a polymer grid comprising a body of electrically conducting organic polymer, said body having an open and porous network morphology and defining an expanded surface area, void-defining porous network interposed between the first and second electrodes, and an active luminescent semiconducting electronic material also interposed between the first and second electrodes which serves to transport electronic charge carriers between the first and second electrodes, the carriers being affected by the polymer grid, such that on applying a turn-on voltage between the first and second electrodes, charge carriers are injected and light is emitted
wherein the active luminescent semiconducting electronic material comprises the compound of claim 1.

18. A light-responsive diode system comprising
a diode comprising:
a conducting first layer having high work function,
a semiconducting second layer in contact with the first layer, the second layer made comprising a compound of claim 1, and
a conducting third layer in contact with the second layer;
a source for applying a reverse bias across the diode;
a source for impinging light upon the diode; and
a source for detecting an electrical current produced by the diode when the reverse bias is applied to the diode and light is impinged upon the diode.

19. A light-responsive diode system comprising
a diode comprising
a conducting first layer having high work function,
a semiconducting second layer in contact with the first layer, the second layer made comprising a compound of claim 1, and
a conducting third layer in contact with the second layer, the third layer comprising an inorganic semiconductor doped to give rise to a conductive state;
a source for applying a reverse bias across the diode;
a source for impinging light upon the diode; and
a source for detecting an electrical current produced by the diode when the reverse bias is applied to the diode and light is impinged upon the diode.

20. A dual function light-emitting, light responsive input-output diode system comprising
a diode comprising
a conducting first layer having high work function,
a semiconducting second layer in contact with the first layer, the second layer made comprising a compound of claim 1, and
a conducting third layer in contact with the second layer;
a source for applying a reverse bias across the diode;
a source for impinging light upon the diode; and
a source for detecting an electrical current produced by the diode when the reverse bias is applied to the diode and light is impinged upon the diode.

21. A dual function light-emitting, light responsive input-output diode system comprising
a diode comprising
a conducting first layer having high work function,
a semiconducting second layer in contact with the first layer, the second layer made comprising a compound of claim 1, and
a conducting third layer in contact with the second layer;
a source for applying a reverse bias across the diode;
a source for impinging an input signal or light upon the diode;
a source for detecting an electrical current produced by the diode when the reverse bias is applied to the input signal of light is impinged upon the diode;
a source for halting the applying of reverse bias; and
a source for applying a positive bias output signal across the diode, said positive bias output signal being adequate to cause the diode to emit an output signal of light.

22. A dual function input-output process employing a light-emitting, light-responsive input-output diode system comprising
a diode comprising
a conducting first layer having high work function,
a semiconducting second layer in contact with the first layer, the second layer made comprising a compound of claim 1, and
a conducting third layer in contact with the second layer; comprising the steps of:
applying a reverse bias across the diode and impinging an input signal of light upon the diode,
detecting as an electrical input signal an electrical current or voltage produced by the diode when the reverse bias is applied to the diode and the input signal of light is impinged upon the diode,
halting the applying of reverse bias, and
applying a positive bias output signal across the diode, said positive bias output signal being adequate to cause the diode to emit an output signal of light in response thereto.

23. An article comprising a unitary solid state source of electromagnetic radiation, said source comprising a layer structure that comprises a multiplicity of layers, including two spaced apart conductor layers with compound of claim 1 therebetween, and further comprising contacts for causing an electrical current to flow between said conductor layers, such that incoherent, electromagnetic radiation of a first wavelength is emitted from said compound of claim 1; characterized in that
the layer structure further comprises an optical waveguide comprising a first and a second cladding region with a core region therebetween, with the optical waveguide disposed such that at least some of said incoherent electromagnetic radiation of the first wavelength is received by the optical waveguide; and
said core region comprises a layer of a second organic material selected to absorb said incoherent electromagnetic radiation of the first wavelength, and to emit coherent electromagnetic radiation of a second wavelength, longer than the first wavelength, in response to said absorbed incoherent electromagnetic radiation.

24. A method comprising the steps of:
providing a conjugated compound comprising at least two covalently bound moieties;
exposing said compound to an energy source for a time and under conditions effective to cause said compound to emit light that has a wavelength of 650-2000 nm; and
determining whether or not said emitted light is of an intensity that is greater than a sum of light emitted by said moieties.
